(19) United States
(12) Patent Application Publication
(10) Pub. No.: US 2013/0231303 A1
(43) Pub. Date: Sep. 5, 2013

(54) 5-HALOGENOPYRAZOLE(THIO)CARBOXAMIDES

(71) Applicants: Juergen Benting, Leichlingen (DE); Pierre Cristau, Lyon (FR); Peter Dahmen, Neuss (DE); Philippe Desbordes, Lyon (FR); Stephanie Gary, Champagne-au-Mont-d'Or (FR); Joerg Greul, Leverkusen (DE); Hiroyuki Hadano, Shimotsuke-Shi (JP); Jan Peter Schmidt, Lyon (FR); Tomoki Tsuchiya, Lyon (FR); Ulrike Wachendorff-Neumann, Neuwied (DE); Lars Rodefeld, Leverkusen (DE)

(72) Inventors: Juergen Benting, Leichlingen (DE); Pierre Cristau, Lyon (FR); Peter Dahmen, Neuss (DE); Philippe Desbordes, Lyon (FR); Stephanie Gary, Champagne-au-Mont-d'Or (FR); Joerg Greul, Leverkusen (DE); Hiroyuki Hadano, Shimotsuke-Shi (JP); Jan Peter Schmidt, Lyon (FR); Tomoki Tsuchiya, Lyon (FR); Ulrike Wachendorff-Neumann, Neuwied (DE); Lars Rodefeld, Leverkusen (DE)

(73) Assignee: Bayer Intellectual Property GmbH, Monheim (DE)

(21) Appl. No.: 13/884,929

(22) PCT Filed: Nov. 14, 2011
(86) PCT No.: PCT/EP11/70038

§ 371 Date: May 10, 2013

(30) Foreign Application Priority Data
Nov. 15, 2010 (EP) 10191265.7

(51) Int. Cl. C07D 409/12; C07F 7/08; A01N 55/00; A01N 43/56

(52) U.S. Cl. CPC C07D 409/12; A01N 43/56; C07F 7/0812; A01N 55/00
USPC 514/63; 548/365.7; 514/406; 548/110

(57) ABSTRACT

The present invention relates to novel 5-halogenopyrazole(thio)carboxamides, their process of preparation, their use as fungicide active agents, particularly in the form of fungicide compositions, and methods for the control of phytopathogenic fungi, notably of plants, using these compounds or compositions.

The present invention relates to novel 5-halogenopyrazole(thio)carboxamides, their process of preparation, their use as fungicide active agents, particularly in the form of fungicide compositions, and methods for the control of phytopathogenic fungi, notably of plants, using these compounds or compositions.

It is already known that numerous carboxamides have fungicidal properties (cf., for example, WO-A 1998/03495, WO-A 1998/03486 and EP-A 0 589 313). Thus, 1-methyl-3-trifluoromethyl-5-fluoro- and 1,3-dimethylfluoropyrazolecarboxamides are already known (WO-A 2006/061215, WO-A 2005/042494, WO-A 2005/042493, WO-A 2008/095890, WO-A 2004/035589, WO-A 2003/074491).

The activity of these compounds is good; however, in some cases, for example at low application rates, it is sometimes unsatisfactory.

This invention now provides novel 1-methyl-3-dihalogenomethyl-5-halogenopyrazole(thio)carboxamides of the formula (I)

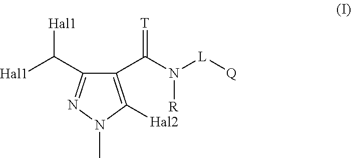

in which T represents an oxygen or sulfur atom
R represents hydrogen, C₁-C₆-alkylsulfonyl, C₁-C₄-alkoxy-C₁-C₄-alkyl, C₁-C₄-haloalkyl-sulfonyl, halo-C₁-C₄-alkoxy-C₁-C₄-alkyl or formyl;
Hal1 and Hal2 independently of one another represent chlorine or fluorine;
Q represents Q¹, Q², Q³, Q⁴, Q⁵, or Q⁶;
L represents

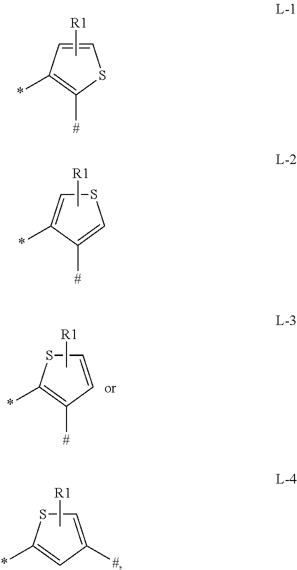

where the bond marked by * is attached to the amide while the bond marked # is attached to Q;
R¹ represents hydrogen, halogen, C₁-C₄-alkyl or C₁-C₄-haloalkyl;
Q¹ represents

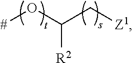

where the bond marked # is attached to L;
R² represents hydrogen, C₁-C₄-alkyl, C₃-C₆-cycloalkyl, C₁-C₄-haloalkyl or optionally substituted phenyl;
Z¹ represents —CR³R⁴R⁵ or —SiR³R⁴R⁵;
s represents 0, 1, 2 or 3;
t represents 0 or 1;
R³ represent hydrogen, cyano, C₁-C₈-alkyl, or C₁-C₆-haloalkyl;
R⁴, R⁵ independently of one another represents hydrogen, C₁-C₈-alkyl, or C₁-C₆-haloalkyl; or
R³ and R⁴ together with the carbon atom to which they are attached form a 3- to 6-membered optionally substituted carbocyclic or heterocyclic saturated or unsaturated ring;
Q² represents

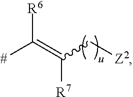

where the bond marked # is attached to L;
R⁶ represents hydrogen, C₁-C₄-alkyl, C₃-C₆-cycloalkyl, C₁-C₄-haloalkyl or optionally substituted phenyl;
R⁷ represents hydrogen, C₁-C₄-alkyl, or C₁-C₄-haloalkyl;
Z² represents hydrogen, —CR⁸R⁹R¹⁰ or —SiR⁸R⁹R¹⁰;
u represents 0, 1 or 2;
R⁸ represent hydrogen, cyano, C₁-C₈-alkyl, or C₁-C₆-haloalkyl; or
R⁷ and R⁸ together with the carbon atoms to which they are attached form a 3- to 6-membered optionally substituted carbocyclic saturated or unsaturated ring;
R⁹, R¹⁰ independently of one another represents hydrogen, C₁-C₈-alkyl, or C₁-C₆-haloalkyl; or
R⁸ and R⁹ together with the carbon atom to which they are attached form a 3- to 6-membered optionally substituted carbocyclic saturated or unsaturated ring;
Q³ represents

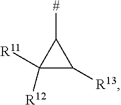

where the bond marked # is attached to L;
R¹¹ represents hydrogen or halogen;
R¹² represents hydrogen or halogen;
R¹³ represents optionally substituted C₂-C₁₂-alkyl, optionally substituted C₂-C₁₂-alkenyl, optionally substituted C₂-C₁₂-alkynyl, optionally substituted C₃-C₁₂-cycloalkyl, optionally substituted phenyl or heterocyclyl;
Q⁴ represents

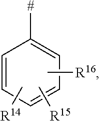

where the bond marked # is attached to L,
R¹⁴, R¹⁵ and R¹⁶ independently of one another represent halogen, cyano, nitro, C₁-C₆-alkyl, C₂-C₆-alkenyl, C₁-C₄-alkoxy, C₁-C₄-alkylsulfanyl, C₁-C₄-alkylsulfonyl, C₃-C₆-cycloalkyl, or represent C₁-C₄-haloalkyl, C₁-C₄-haloalkoxy, C₁-C₄-haloalkylsulfanyl or C₁-C₄-halo-alkylsulfonyl having in each case 1 to 5 halogen atoms;
Q⁵ represents

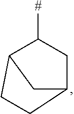

where the bond marked # is attached to L;
Q⁶ represents

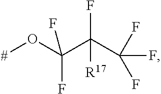

where R¹⁷ represents hydrogen or fluorine;
where, unless indicated otherwise, a group or a substituent which is substituted according to the invention is substituted by one or more group selected in the list consisting of halogen; nitro, cyano, C₁-C₁₂-alkyl; C₁-C₆-haloalkyl having 1 to 9 identical or different halogen atoms; C₁-C₆-alkoxy; C₁-C₆-haloalkoxy having 1 to 9 identical or different halogen atoms; C₁-C₆-alkylsulfanyl; C₁-C₆-haloalkylsulfanyl having 1 to 9 identical or different halogen atoms; C₁-C₆-alkylsulfonyl; C₁-C₆-haloalkylsulfonyl having 1 to 9 identical or different halogen atoms; C₂-C₁₂-alkenyl; C₂-C₁₂-alkynyl; C₃-C₇-cycloalkyl; phenyl; tri(C₁-C₈)alkylsilyl; tri(C₁-C₈)alkylsilyl-C₁-C₈-alkyl;

The formula (I) provides a general definition of the 1-methyl-3-dihalogeno-5-halogenopyrazole(thio)carboxamides according to the invention. Preferred radical definitions for the formulae shown above and below are given below. These definitions apply to the end products of the formula (I) and likewise to all intermediates.
T preferably represents an oxygen atom
R preferably represents hydrogen, C₁-C₄-alkylsulfonyl, C₁-C₃-alkoxy-C₁-C₃-alkyl, C₁-C₄-haloalkylsulfonyl, halo-C₁-C₃-alkoxy-C₁-C₃-alkyl having in each case 1 to 9 fluorine, chlorine and/or bromine atoms; or formyl.
R particularly preferably represents hydrogen, methylsulfonyl, ethylsulfonyl, n- or isopropyl-sulfonyl, n-, iso-, sec- or tert-butylsulfonyl, methoxymethyl, methoxyethyl, ethoxymethyl, ethoxyethyl, trifluoromethylsulfonyl, trifluoromethoxymethyl; or formyl.
R very particularly preferably represents hydrogen, methoxymethyl, or formyl.
Hal1 preferably represents chlorine.
Hal1 preferably represents fluorine.
Hal2 preferably represents chlorine.
Hal2 preferably represents fluorine.
L moreover preferably represents L-1
L moreover preferably represents L-2
L moreover preferably represents L-3.
R¹ preferably represents hydrogen or chlorine
Z¹ preferably represents —CR³R⁴R⁵.
Z¹ moreover preferably represents —SiR³R⁴R⁵.
s preferably represents 0.
s preferably represents 1.
s moreover preferably represents 2.
s particularly preferably represents 1.
t preferably represents 0.
t preferably represents 1.
R² preferably represents hydrogen, fluorine, chlorine, methyl, ethyl, n- or isopropyl, or represents methyl, ethyl, n- or isopropyl, n-, iso-, sec- or tert-butyl, each of which is mono- or polysubstituted by identical or different substituents from the group consisting of fluorine, chlorine and bromine
R² particularly preferably represents hydrogen, fluorine, chlorine, methyl, ethyl, trifluoromethyl, difluoromethyl, fluoromethyl, trichloromethyl, dichloromethyl, chloromethyl, chlorofluoromethyl, fluorodichloromethyl, difluorochloromethyl, pentafluoroethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2-chloro-2,2-difluoroethyl, 2-dichloro-2-fluoroethyl, 2,2,2-tri-chloroethyl, 1-chlorobutyl, heptafluoro-n-propyl or heptafluoroisopropyl.
R² very particularly preferably represents hydrogen, methyl or ethyl.
R² especially preferably represents hydrogen or methyl.
R³ preferably represents fluorine, chlorine, bromine, methyl, ethyl, n- or isopropyl, n-, iso-, sec- or tert-butyl or represents methyl, ethyl, n- or isopropyl, n-, iso-, sec- or tert-butyl, each of which is mono- or polysubstituted by identical or different substituents from the group consisting of fluorine, chlorine and bromine
R³ particularly preferably represents fluorine, chlorine, methyl, ethyl, n- or isopropyl, n-, iso-, sec- or tert-butyl, trifluoromethyl, difluoromethyl, fluoromethyl, trichloromethyl, dichloromethyl, chloromethyl, chlorofluoromethyl, fluorodichloromethyl, difluorochloro-methyl, pentafluoroethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2-chloro-2,2-difluoroethyl, 2-dichloro-2-fluoroethyl, 2,2,2-trichloroethyl, 1-chlorobutyl, heptafluoro-n-propyl or heptafluoroisopropyl.
R³ very particularly preferably represents chlorine, methyl, ethyl, isopropyl or trifluoromethyl.
R⁴ preferably represents fluorine, chlorine, bromine, methyl, ethyl, n- or isopropyl, n-, iso-, sec- or tert-butyl or represents methyl, ethyl, n- or isopropyl, n-, iso-, sec- or tert-butyl, each of which is mono- or polysubstituted by identical or different substituents from the group consisting of fluorine, chlorine and bromine
R⁴ particularly preferably represents fluorine, chlorine, methyl, ethyl, n- or isopropyl, n-, iso-, sec- or tert-butyl, trifluoromethyl, difluoromethyl, fluoromethyl, trichloromethyl, dichloromethyl, chloromethyl, chlorofluoromethyl, fluorodichloromethyl, difluorochloro-methyl, pentafluoroethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2-chloro-2,2-difluoroethyl, 2-dichloro-2-fluoroethyl, 2,2,2-trichloroethyl, 1-chlorobutyl, heptafluoro-n-propyl or heptafluoroisopropyl.
R⁴ very particularly preferably represents chlorine, methyl, ethyl, isopropyl or trifluoromethyl.

In a particular embodiment, R³ and R⁴ together with the carbon atom to which they are attached preferably form a 3- to 6-membered carbocyclic or heterocyclic saturated or unsaturated ring which is optionally substituted by halogen, methyl, ethyl, methoxy, trifluoromethyl or trifluoromethoxy,
R³ and R⁴ together with the carbon atom to which they are attached particularly preferably form a 3-, 5- or 6-membered carbocyclic saturated ring which is optionally substituted by methyl, ethyl or trifluoromethyl,
R³ and R⁴ moreover together with the carbon atom to which they are attached very particularly preferably form a 6-membered carbocyclic unsaturated ring which is optionally substituted by halogen, methyl, ethyl, methoxy, trifluoromethyl or trifluoromethoxy.
R⁵ preferably represents hydrogen, fluorine, chlorine, bromine, methyl, ethyl, n- or isopropyl, n-, iso-, sec- or tert-butyl or represents methyl, ethyl, n- or isopropyl, n-, iso-, sec- or tert-butyl, each of which is mono- or polysubstituted by identical or different substituents from the group consisting of fluorine, chlorine and bromine
R⁵ particularly preferably represents hydrogen, fluorine, chlorine, methyl, ethyl, n- or isopropyl, iso-, sec- or tert-butyl, trifluoromethyl, difluoromethyl, fluoromethyl, trichloromethyl, dichloromethyl, chloromethyl, chlorofluoromethyl, fluorodichloromethyl, difluorochloro-methyl, pentafluoroethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2-chloro-2,2-difluoroethyl, 2-dichloro-2-fluoroethyl, 2,2,2-trichloroethyl, 1-chlorobutyl, heptafluoro-n-propyl or heptafluoroisopropyl.
R⁵ very particularly preferably represents hydrogen, chlorine, methyl, ethyl, isopropyl or trifluoromethyl.
Z² preferably represents —CR⁸R⁹R¹⁰.
Z² moreover preferably represents —SiR⁸R⁸R¹⁰.
u preferably represents 0.
u preferably represents 1.
u particularly preferably represents 1.
R⁶ preferably represents hydrogen, methyl or ethyl.
R⁷ preferably represents hydrogen, methyl or ethyl.
R⁸ preferably represents fluorine, chlorine, bromine, methyl, ethyl, n- or isopropyl, n-, iso-, sec- or tert-butyl or represents methyl, ethyl, n- or isopropyl, n-, iso-, sec- or tert-butyl, each of which is mono- or polysubstituted by identical or different substituents from the group consisting of fluorine, chlorine and bromine
R⁸ particularly preferably represents fluorine, chlorine, methyl, ethyl, n- or isopropyl, n-, iso-, sec- or tert-butyl, trifluoromethyl, difluoromethyl, fluoromethyl, trichloromethyl, dichloromethyl, chloromethyl, chlorofluoromethyl, fluorodichloromethyl, difluorochloro-methyl, pentafluoroethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2-chloro-2,2-difluoroethyl, 2-dichloro-2-fluoroethyl, 2,2,2-trichloroethyl, 1-chlorobutyl, heptafluoro-n-propyl or heptafluoroisopropyl.
R⁸ very particularly preferably represents chlorine, methyl, ethyl, isopropyl or trifluoromethyl.
R⁹ preferably represents fluorine, chlorine, bromine, methyl, ethyl, n- or isopropyl, n-, iso-, sec- or tert-butyl or represents methyl, ethyl, n- or isopropyl, n-, iso-, sec- or tert-butyl, each of which is mono- or polysubstituted by identical or different substituents from the group consisting of fluorine, chlorine and bromine
R⁹ particularly preferably represents fluorine, chlorine, methyl, ethyl, n- or isopropyl, n-, iso-, sec- or tert-butyl, trifluoromethyl, difluoromethyl, fluoromethyl, trichloromethyl, dichloromethyl, chloromethyl, chlorofluoromethyl, fluorodichloromethyl, difluorochloro-methyl, pentafluoroethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2-chloro-2,2-difluoroethyl, 2-dichloro-2-fluoroethyl, 2,2,2-trichloroethyl, 1-chlorobutyl, heptafluoro-n-propyl or heptafluoroisopropyl.
R⁹ very particularly preferably represents chlorine, methyl, ethyl, isopropyl or trifluoromethyl.

In a particular embodiment, R⁸ and R⁹ together with the carbon atom to which they are attached preferably form a 3- to 6-membered carbocyclic or heterocyclic saturated or unsaturated ring which is optionally substituted by halogen, methyl, ethyl, methoxy, trifluoromethyl or trifluoromethoxy,
R⁸ and R⁹ moreover together with the carbon atom to which they are attached particularly preferably form a 3-, 5- or 6-membered carbocyclic saturated ring which is optionally substituted by methyl, ethyl or trifluoromethyl,
R⁸ and R⁹ moreover together with the carbon atom to which they are attached very particularly preferably form a 6-membered carbocyclic unsaturated ring which is optionally substituted by halogen, methyl, ethyl, methoxy, trifluoromethyl or trifluoromethoxy.
R¹⁰ preferably represents hydrogen, fluorine, chlorine, bromine, methyl, ethyl, n- or isopropyl, n-, iso-, sec- or tert-butyl or represents methyl, ethyl, n- or isopropyl, n-, iso-, sec- or tert-butyl, each of which is mono- or polysubstituted by identical or different substituents from the group consisting of fluorine, chlorine and bromine
R¹⁰ particularly preferably represents hydrogen, fluorine, chlorine, methyl, ethyl, n- or isopropyl, iso-, sec- or tert-butyl, trifluoromethyl, difluoromethyl, fluoromethyl, trichloromethyl, dichloromethyl, chloromethyl, chlorofluoromethyl, fluorodichloromethyl, difluorochloro-methyl, pentafluoroethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2-chloro-2,2-difluoroethyl, 2-dichloro-2-fluoroethyl, 2,2,2-trichloroethyl, 1-chlorobutyl, heptafluoro-n-propyl or heptafluoroisopropyl.
R¹⁰ very particularly preferably represents hydrogen, chlorine, methyl, ethyl, isopropyl or trifluoromethyl.
R¹¹ and R¹² independently of one another preferably represent hydrogen, fluorine, chlorine or bromine;
R¹¹ and R¹² independently of one another particularly preferably represent hydrogen or fluorine;
R¹¹ and R¹² very particularly preferably represent hydrogen;
R¹³ preferably represents C₂-C₆-alkyl, optionally substituted C₃-C₈-cycloalkyl, optionally substituted phenyl, pyridyl, thienyl or furyl;
R¹³ particularly preferably represents ethyl, n-propyl, isopropyl, n-butyl, sec-butyl, tert-butyl, n-pentyl, neopentyl, isopentyl, n-hexyl, isohexyl, n-heptyl, cyclopropyl, cyclobutyl, cyclopentyl, cyclohexyl, cycloheptyl, cyclooctyl, a-methylcyclopropyl, 2-, 3- or 4-halo-substituted phenyl, 2-thienyl, 3-thienyl or 2-furyl;
R¹³ very particularly preferably represents ethyl, n-propyl, isopropyl, n-butyl, sec-butyl, tert-butyl, cyclopropyl, cyclobutyl, cyclopentyl, cyclohexyl, a-methylcyclopropyl, 4-fluorophenyl, 4-bromophenyl, 4-chlorophenyl, 2-thienyl, 3-thienyl or 2-furyl;
R¹⁴, R¹⁵ and R¹⁶ independently of one another preferably represent fluorine, chlorine, bromine, cyano, nitro, methyl, ethyl, n- or isopropyl, n-, s- or t-butyl, methoxy, ethoxy, methylsulfanyl, ethylsulfanyl, n- or isopropylsulfanyl, cyclopropyl, trifluoromethyl, trichloromethyl, trifluoro-ethyl, difluoromethoxy, trifluoromethoxy, difluorochloromethoxy, trifluoroethoxy, difluor-omethylsulfanyl, difluorochloromethylsulfanyl or trifluoromethylsulfanyl;
R¹⁴, R⁵ and R¹⁶ independently of one another particularly preferably represent fluorine, chlorine, bromine, methyl, trifluoromethyl, difluoromethoxy or trifluoromethoxy;
R¹⁴, R¹⁵ and R¹⁶ independently of one another very particularly preferably represent fluorine, chlorine, bromine or trifluoromethyl.

Preference is given to compounds of the formula (I) in which all radicals in each case have the preferred meanings mentioned above.

Particular preference is given to compounds of the formula (I) in which all radicals in each case have the particularly preferred meanings mentioned above.

Very particular preference is given to compounds of the formula (I) in which all radicals in each case have the very particularly preferred meanings mentioned above.

The following groups of novel (thio)carboxamides of the formula

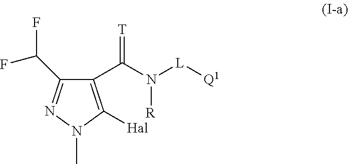

in which T, R, L, Q¹ have the meanings given above and Hal represents fluorine or chlorine;

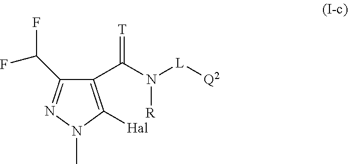

in which T, R, L, Q² have the meanings given above and Hal represents fluorine or chlorine;

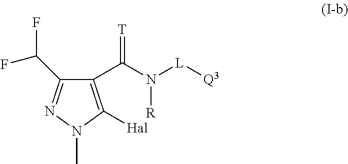

in which T, R, L, Q³ have the meanings given above and Hal represents fluorine or chlorine;

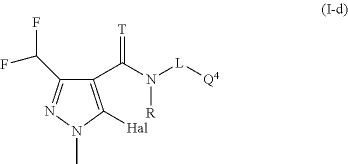

in which T, R, L, Q⁴ have the meanings given above and Hal represents fluorine or chlorine;

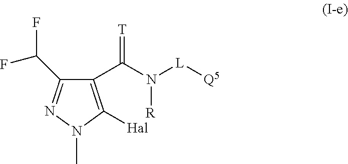

in which T, R, L, Q⁵ have the meanings given above and Hal represents fluorine or chlorine;

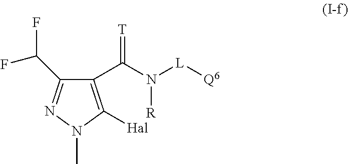

in which T, R, L, Q⁶ have the meanings given above and Hal represents fluorine or chlorine;
   are preferred and in each case to be understood as a subset of the compounds of the formula (I) mentioned above.

The following groups of novel carboxamides of the formulae

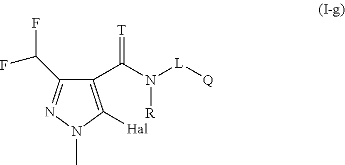

in which T, R, L and Q have the meanings given above and Hal represents fluorine;

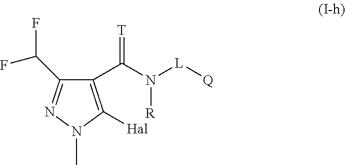

in which T, R, L and Q have the meanings given above and Hal represents chlorine;
   are preferred and in each case to be understood as a subset of the compounds of the formula (I) mentioned above.

Very particular special preference is given to compounds of the formula (I) where
T represents oxygen
R represents hydrogen;
Hal represents chlorine or fluorine;
Hal1 and Hal2 independently of one another represent chlorine or fluorine;
Q represents Q¹, Q², Q³, Q⁴, Q⁵, or Q⁶;

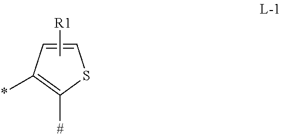

L represents,
where the bond marked * is attached to the amide while the bond marked # is attached to Q;
R¹ represents hydrogen;
Q¹ represents

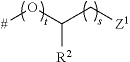

where the bond marked # is attached to L,
R² represents hydrogen, methyl or ethyl,
Z¹ represents —CR³R⁴R⁵ or —SiR³R⁴R⁵;
s represents 0, 1, 2 or 3;
t represents 0,
R³, R⁴, R⁵ independently of one another represent hydrogen or methyl;
Q² represents

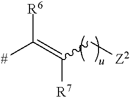

where the bond marked # is attached to L,
R⁶ represents hydrogen or methyl,
R⁷ represents hydrogen or methyl,
Z² represents —CR⁸R⁹R¹⁰ or —SiR⁸R⁹R¹⁰;
u represents 0, 1 or 2;
R⁸, R⁹, R¹⁰ independently of one another represent hydrogen or methyl;
Q³ represents

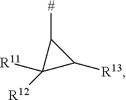

where the bond marked # is attached to L;
R¹¹ and R¹² represent hydrogen;
R¹³ represents cyclopropyl;
Q⁴ represents

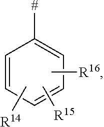

where the bond marked # is attached to L;
R¹⁴, R¹⁵ and R¹⁶ independently of one another represent fluorine, chlorine, bromine or trifluoromethyl;
Q⁵ represents

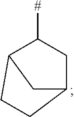

Q⁶ represents

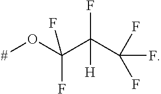

Saturated or unsaturated hydrocarbon radicals, such as alkyl or alkenyl, can in each case be straight-chain or branched as far as this is possible, including in combination with heteroatoms, such as, for example, in alkoxy.

Optionally substituted radicals can be mono- or polysubstituted, where in the case of polysubstitution the substituents can be identical or different. Thus, the definition dialkylamino also includes an amino group which is unsymmetrically substituted by alkyl, such as, for example, methylethylamino.

Halogen-substituted radicals, such as, for example, halogenoalkyl, are mono- or polyhalogenated. In the case of polyhalogenation, the halogen atoms can be identical or different. Halogen denotes fluorine, chlorine, bromine and iodine, in particular fluorine, chlorine and bromine.

The compounds according to the invention may, if appropriate, be present as mixtures of various possible isomeric forms, in particular stereoisomers such as, for example, E and Z, threo and erythro, and also optical isomers, and, if appropriate, also of tautomers. What is claimed are both the E and the Z isomers, and also the threo and erythro, and also the optical isomers, any mixtures of these isomers, and also the possible tautomeric forms.

In the variations according to the invention of Q, up to two stereocentres (*) are present. What is claimed are all diastereomers and enantiomers, in particular of the following radicals:

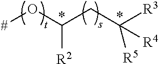

The abovementioned general or preferred radical definitions or illustrations can be combined as desired between the respective ranges and preferred ranges. They apply both to the end products and, correspondingly, to precursors and intermediates. In particular the compounds mentioned in groups (I-a) to (I-h) can be combined both with the general and the preferred, particularly preferred, etc., meanings, where here, too, in each case all combinations between the preferred ranges are possible.

Finally, it has been found that the novel 1-methyl-3-dihalogenomethyl-5-halogenopyrazole (thio)carboxamides of the formula (I) have very good microbicidal properties and can be used for controlling unwanted microorganisms both in crop protection and in the protection of materials.

Furthermore, it has been found that 1-methyl-3-dihalogenomethyl-5-halogenopyrazolecarboxamides of the formula (I) wherein T represents an oxygen atom are obtained when carboxylic acid derivatives of the formula (II)

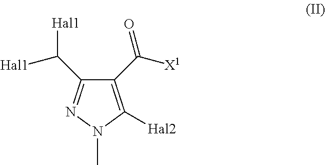

in which
X¹ represents halogen or hydroxyl,
Hal1 and Hal2 independently of one another represent chlorine or fluorine;
are reacted with amine derivatives of the formula (III)

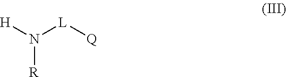

in which R, L and Q have the meanings given above,
if appropriate in the presence of a catalyst, if appropriate in the presence of a condensing agent, if appropriate in the presence of an acid binder and if appropriate in the presence of a diluent.

This process for synthesizing such amides of the formula (I) has already been described sufficiently: WO-A 2006/061215, WO-A 2005/042494, WO-A 2005/042493, WO-A 2008/095890, WO-A:

The acids and acid halides of the formula (II) used

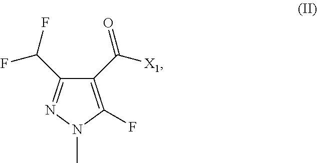

in which
X¹ represents halogen or hydroxyl,
are novel and are synthesized as described below:

Preference is given to compounds of the formula (II) where X¹ represents hydroxyl, chlorine or fluorine.

A further aspect of the invention comprises the process P1 according to the invention for synthesizing the acid chlorides of the formula (II), as shown in the reaction scheme below:

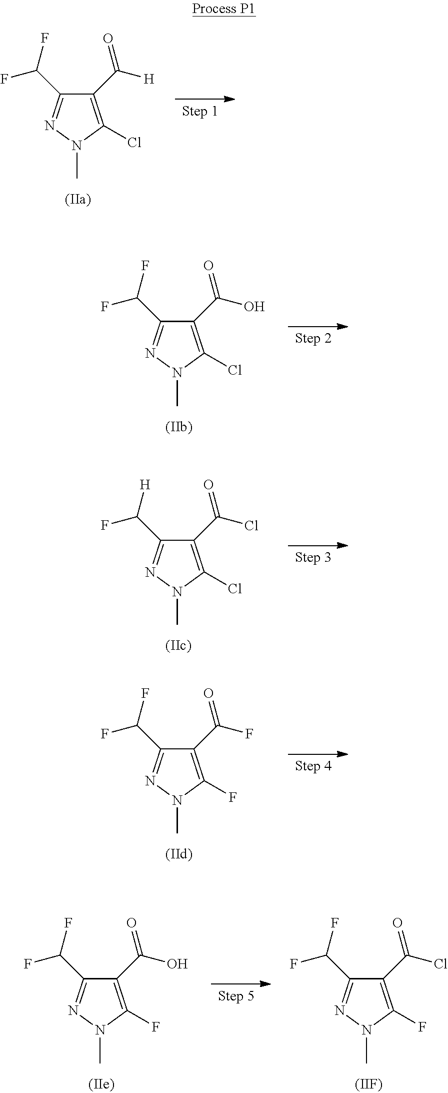

The compound of the formula (IIa), 5-chloro-3-(difluoromethyl)-1-methyl-1H-pyrazole-4-carbaldehyde, is known from WO-2004/014138 (Example 35).

Step 1 in process P1 according to the invention is carried out in the presence of an oxidizing agent and, if required, in the presence of a solvent.

Steps 2 and 5 in process P1 according to the invention are carried out in the presence of an acid halide former and, if required, in the presence of a solvent.

Step 3 in process P1 according to the invention is carried out in the presence of a fluorinating agent and, if required, in the presence of a solvent.

Step 4 in process P1 according to the invention is carried out in the presence of an acid or a base and, if required, in the presence of a solvent.

Suitable oxidizing agents for carrying out step 1 of process P1 according to the invention are all inorganic and organic oxidizing agents customarily used for such reactions.

Preference is given to benzyltriethylammonium permanganate, bromine, chlorine, m-chloroperbenzoic acid, chromic acid, chromium(VI) oxide, hydrogen peroxide, hydrogen peroxide/boron trifluoride, hydrogen peroxide/urea adduct, 2-hydroxyperoxyhexafluoro-2-propanol, iodine, perbenzoic acid, peroxyacetyl nitrate, potassium permanganate, potassium ruthenate, pyridinium dichromate, ruthenium(VIII) oxide, silver(I) oxide, silver(II) oxide, silver nitrite, sodium chlorite, sodium hypochlorite, 2,2,6,6-tetramethylpiperidine-1-oxyl.

Suitable acid halide formers for step 2 and step 5 of process P1 according to the invention are all organic and inorganic acid halide formers customarily used for such reactions.

Preference is given to phosgene, phosphorus trichloride, phosphorus pentachloride, phosphorus oxytrichloride; thionyl chloride; or carbon tetrachloride/triphenylphosphine.

Suitable fluorinating agents for step 3 of process P1 according to the invention are those which are customarily used for such reactions.

Preference is given to caesium fluoride; potassium fluoride; potassium fluoride/calcium difluoride and also tetrabutylammonium fluoride.

Suitable solvents for steps 1 to 5 of process P1 according to the invention are all customary inert organic solvents.

Preference is given to using optionally halogenated aliphatic, alicyclic or aromatic hydrocarbons, such as petroleum ether, hexane, heptane, cyclohexane, methylcyclohexane, benzene, toluene, xylene or decaline; chlorobenzene, dichlorobenzene, dichloromethane, chloroform, carbon tetrachloride, dichloroethane or trichloroethane; various ethers, such as diethyl ether, cyclopentyl methyl ether, diisopropyl ether, methyl t-butyl ether, methyl t-amyl ether, dioxane, tetrahydrofuran, 2-methyltetrahydrofuran, 1,2-dimethoxyethane, 1,2-diethoxyethane or anisole; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate, sulfoxides, such as dimethyl sulfoxide, or sulfones, such as sulfolane.

When carrying out steps 1 to 5 of process P1 according to the invention, the reaction temperatures can be varied over a relatively wide range. In general, the steps are carried out at temperatures of 0° C. to 160° C., preferably at temperatures from 0° C. to 120° C.

As a means for controlling the temperature in process P1 according to the invention, it is also possible to use microwave technology.

Unless indicated otherwise, all steps of process P1 according to the invention are generally carried out under atmospheric pressure. However, it is also possible to operate under elevated or reduced pressure—in general between 0.1 and 10 bar.

For carrying out step 1 of process P1 according to the invention, in general an excess of oxidizing agent is employed per mole of the aldehyde derivative of the formula (IIa). However, it is also possible to employ the components in other ratios.

For carrying out steps 2 and 5 of process P1 according to the invention, in general an excess of acid halide former is employed per mole of the carboxylic acid derivative of the formula (IIb) or (IIe). However, it is also possible to employ the components in other ratios.

For carrying out step 3 of process P1 according to the invention, in general an excess of fluorinating agent is employed per mole of the acid halide derivative of the formula (IIc). However, it is also possible to employ the components in other ratios.

For carrying out step 4 of process P1 according to the invention, in general an excess of acid or base is employed per mole of the acid fluoride derivative of the formula (IId). However, it is also possible to employ the components in other ratios.

A further aspect of the invention comprises the process P2 according to the invention for synthesizing 1-methyl-3-dihalogenomethyl-5-halogenopyrazolethiocarboxamides of the formula (I) wherein T represents an sulfur atom, as shown in the reaction scheme below:

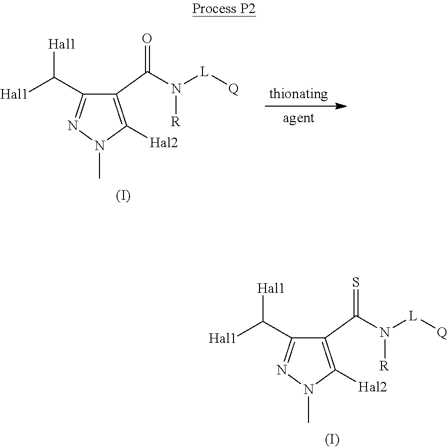

wherein Hal1, Hal2, R, L and Q are as herein-defined,
in the optionally presence of a catalytic or stoichiometric or excess amount, quantity of a base such as an inorganic and organic base. Preference is given to using alkali metal carbonates, such as sodium carbonate, potassium carbonate, potassium bicarbonate, sodium bicarbonate; heterocyclic aromatic bases, such as pyridine, picoline, lutidine, collidine; and also tertiary amines, such as trimethylamine, triethylamine, tributylamine, N,N-dimethylaniline, N,N-dimethylaminopyridine or N-methyl-piperidine.

Process P2 according to the invention is performed in the presence of a thionating agent.

Starting amide derivatives of formula (I) can be prepared as previously described.

Suitable thionating agents for carrying out process P2 according to the invention can be sulfur (S), sulfhydric acid (H₂S), sodium sulfide (Na₂S), sodium hydrosulfide (NaHS), boron trisulfide (B₂S₃), bis(diethylaluminium) sulfide ((AlEt₂)₂S), ammonium sulfide ((NH₄)₂S), phosphorous pentasulfide (P₂S₅), Lawesson's reagent (2,4-bis(4-methoxyphenyl)-1,2,3,4-dithiadiphosphetane 2,4-disulfide) or a polymer-supported thionating reagent such as described in Journal of the Chemical Society, Perkin 1 (2001), 358.

The compounds according to the invention can be synthesized according to the process described above. Based on his expert knowledge, the person skilled in the art is able to modify the preparation processes for the compounds according to the invention in a suitable manner.

Certain amines of the formula (III) used are already known such as:
2-(4-methylpentan-2-yl)thiophene-3-amine (EP-A 1 036 793, EP-A 0 737 682).

The compound according to the present invention can be prepared according to the general processes of preparation described above. It will nevertheless be understood that, on the basis of his general knowledge and of available publications, the skilled worker will be able to adapt this method according to the specifics of each of the compounds, which it is desired to synthesize.

In a further aspect, the present invention also relates to a fungicide composition comprising an effective and non-phytotoxic amount of an active compound of formula (I).

The expression “effective and non-phytotoxic amount” means an amount of composition according to the invention that is sufficient to control or destroy the fungi present or liable to appear on the cropsand that does not entail any appreciable symptom of phytotoxicity for the said crops. Such an amount can vary within a wide range depending on the fungus to be controlled, the type of crop, the climatic conditions and the compounds included in the fungicide composition according to the invention. This amount can be determined by systematic field trials that are within the capabilities of a person skilled in the art.

Thus, according to the invention, there is provided a fungicide composition comprising, as an active ingredient, an effective amount of a compound of formula (I) as herein defined and an agriculturally acceptable support, carrier or filler.

According to the invention, the term “support” denotes a natural or synthetic, organic or inorganic compound with that the active compound of formula (I) is combined or associated to make it easier to apply, notably to the parts of the plant. This support is thus generally inert and should be agriculturally acceptable. The support can be a solid or a liquid. Examples of suitable supports include clays, natural or synthetic silicates, silica, resins, waxes, solid fertilisers, water, alcohols, in particular butanol, organic solvents, mineral and plant oils and derivatives thereof. Mixtures of such supports can also be used.

The composition according to the invention can also comprise additional components. In particular, the composition can further comprise a surfactant. The surfactant can be an emulsifier, a dispersing agent or a wetting agent of ionic or non-ionic type or a mixture of such surfactants. Mention can be made, for example, of polyacrylic acid salts, lignosulfonic acid salts, phenolsulfonic or naphthalenesulfonic acid salts, polycondensates of ethylene oxide with fatty alcohols or with fatty acids or with fatty amines, substituted phenols (in particular alkylphenols or arylphenols), salts of sulfosuccinic acid esters, taurine derivatives (in particular alkyl taurates), phosphoric esters of polyoxyethylated alcohols or phenols, fatty acid esters of polyolsand derivatives of the above compounds containing sulfate, sulfonate and phosphate functions. The presence of at least one surfactant is generally essential when the active compound and/or the inert support are water-insoluble and when the vector agent for the application is water. Preferably, surfactant content can be comprised from 5% to 40% by weight of the composition.

Optionally, additional components can also be included, e.g. protective colloids, adhesives, thickeners, thixotropic agents, penetration agents, stabilisers, sequestering agents. More generally, the active compounds can be combined with any solid or liquid additive, that complies with the usual formulation techniques.

In general, the composition according to the invention can contain from 0.05 to 99% by weight of active compound, preferably 10 to 70% by weight.

Compositions according to the invention can be used in various forms such as aerosol dispenser, capsule suspension, cold fogging concentrate, dustable powder, emulsifiable concentrate, emulsion oil in water, emulsion water in oil, encapsulated granule, fine granule, flowable concentrate for seed treatment, gas (under pressure), gas generating product, granule, hot fogging concentrate, macrogranule, microgranule, oil dispersible powder, oil miscible flowable concentrate, oil miscible liquid, paste, plant rodlet, powder for dry seed treatment, seed coated with a pesticide, soluble concentrate, soluble powder, solution for seed treatment, suspension concentrate (flowable concentrate), ultra low volume (ULV) liquid, ultra low volume (ULV) suspension, water dispersible granules or tablets, water dispersible powder for slurry treatment, water soluble granules or tablets, water soluble powder for seed treatment and wettable powder. These compositions include not only compositions that are ready to be applied to the plant or seed to be treated by means of a suitable device, such as a spraying or dusting device, but also concentrated commercial compositions that must be diluted before application to the crop.

The compounds according to the invention can also be mixed with one or more insecticide, fungicide, bactericide, attractant, acaricide or pheromone active substance or other compounds with biological activity. The mixtures thus obtained have normally a broadened spectrum of activity. The mixtures with other fungicide compounds are particularly advantageous.

Examples of suitable fungicide mixing partners can be selected in the following lists:

(1) Inhibitors of the ergosterol biosynthesis, for example (1.1) aldimorph (1704-28-5), (1.2) azaconazole (60207-31-0), (1.3) bitertanol (55179-31-2), (1.4) bromuconazole (116255-48-2), (1.5) cyproconazole (113096-99-4), (1.6) diclobutrazole (75736-33-3), (1.7) difenoconazole (119446-68-3), (1.8) diniconazole (83657-24-3), (1.9) diniconazole-M (83657-18-5), (1.10) dodemorph (1593-77-7), (1.11) dodemorph acetate (31717-87-0), (1.12) epoxiconazole (106325-08-0), (1.13) etaconazole (60207-93-4), (1.14) fenarimol (60168-88-9), (1.15) fenbuconazole (114369-43-6), (1.16) fenhexamid (126833-17-8), (1.17) fenpropidin (67306-00-7), (1.18) fenpropimorph (67306-03-0), (1.19) fluquinconazole (136426-54-5), (1.20) flurprimidol (56425-91-3), (1.21) flusilazole (85509-19-9), (1.22) flutriafol (76674-21-0), (1.23) furconazole (112839-33-5), (1.24) furconazole-cis (112839-32-4), (1.25) hexaconazole (79983-71-4), (1.26) imazalil (60534-80-7), (1.27) imazalil sulfate (58594-72-2), (1.28) imibenconazole (86598-92-7), (1.29) ipconazole (125225-28-7), (1.30) metconazole (125116-23-6), (1.31) myclobutanil (88671-89-0), (1.32) naftifine (65472-88-0), (1.33) nuarimol (63284-71-9), (1.34) oxpoconazole (174212-12-5), (1.35) paclobutrazol (76738-62-0), (1.36) pefurazoate (101903-30-4), (1.37) penconazole (66246-88-6), (1.38) piperalin (3478-94-2), (1.39) prochloraz (67747-09-5), (1.40) propiconazole (60207-90-1), (1.41) prothioconazole (178928-70-6), (1.42) pyributicarb (88678-67-5), (1.43) pyrifenox (88283-41-4), (1.44) quinconazole (103970-75-8), (1.45) simeconazole (149508-90-7), (1.46) spiroxamine (118134-30-8), (1.47) tebuconazole (107534-96-3), (1.48) terbinafine (91161-71-6), (1.49) tetraconazole (112281-77-3), (1.50) triadimefon (43121-43-3), (1.51) triadimenol (89482-17-7), (1.52) tridemorph (81412-43-3), (1.53) triflumizole (68694-11-1), (1.54) triforine (26644-46-2), (1.55) triticonazole (131983-72-7), (1.56) uniconazole (83657-22-1), (1.57) uniconazole-p (83657-17-4), (1.58) viniconazole (77174-66-4), (1.59) voriconazole (137234-62-9), (1.60) 1-(4-chlorophenyl)-2-(1H-1,2,4-triazol-1-yl)cycloheptanol (129586-32-9), (1.61) methyl 1-(2,2-dimethyl-2,3-dihydro-1H-inden-1-yl)-1H-imidazole-5-carboxylate (110323-95-0), (1.62) N′-{5-(difluoromethyl)-2-methyl-4-[3-(trimethylsilyl)propoxy]phenyl}-N-ethyl-N-methylimidoformamide, (1.63) N-ethyl-N-methyl-N′-{2-methyl-5-(trifluoromethyl)-4-[3-(trimethylsilyl)propoxy]phenyl}imidoformamide and (1.64) O-[1-(4-methoxyphenoxy)-3,3-dimethylbutan-2-yl]1H-imidazole-1-carbothioate (111226-71-2).

(2) inhibitors of the respiratory chain at complex I or II, for example (2.1) bixafen (581809-46-3), (2.2) boscalid (188425-85-6), (2.3) carboxin (5234-68-4), (2.4) diflumetorim (130339-07-0), (2.5) fenfuram (24691-80-3), (2.6) fluopyram (658066-35-4), (2.7) flutolanil (66332-96-5), (2.8) fluxapyroxad (907204-31-3), (2.9) furametpyr (123572-88-3), (2.10) furmecyclox (60568-05-0), (2.11) isopyrazam (mixture of syn-epimeric racemate 1RS,4SR,9RS and anti-epimeric racemate 1RS,4SR,9SR) (881685-58-1), (2.12) isopyrazam (anti-epimeric racemate 1RS,4SR,9SR), (2.13) isopyrazam (anti-epimeric enantiomer 1R,4S,9S), (2.14) isopyrazam (anti-epimeric enantiomer 1S,4R,9R), (2.15) isopyrazam (syn epimeric racemate 1RS,4SR,9RS), (2.16) isopyrazam (syn-epimeric enantiomer 1R,4S,9R), (2.17) isopyrazam (syn-epimeric enantiomer 1S,4R,9S), (2.18) mepronil (55814-41-0), (2.19) oxycarboxin (5259-88-1), (2.20) penflufen (494793-67-8), (2.21) penthiopyrad (183675-82-3), (2.22) sedaxane (874967-67-6), (2.23) thifluzamide (130000-40-7), (2.24) 1-methyl-N-[2-(1,1,2,2-tetrafluoroethoxy)phenyl]-3-(trifluoromethyl)-1H-pyrazole-4-carboxamide, (2.25) 3-(difluoromethyl)-1-methyl-N-[2-(1,1,2,2-tetrafluoroethoxy)phenyl]-1H-pyrazole-4-carboxamide, (2.26) 3-(difluoromethyl)-N-[4-fluoro-2-(1,1,2,3,3,3-hexafluoropropoxy)phenyl]-1-methyl-1H-pyrazole-4-carboxamide, (2.27) N-[1-(2,4-dichlorophenyl)-1-methoxypropan-2-yl]-3-(difluoromethyl)-1-methyl-1H-pyrazole-4-carboxamide (1092400-95-7) (WO 2008148570), (2.28) 5,8-difluoro-N-[2-(2-fluoro-4-{[4-(trifluoromethyl)pyridin-2-yl]oxy}phenyl)ethyl]quinazolin-4-amine (1210070-84-0) (WO2010025451), (2.29) N-[9-(dichloromethylene)-1,2,3,4-tetrahydro-1,4-methanonaphthalen-5-yl]-3-(difluoromethyl)-1-methyl-1H-pyrazole-4-carboxamide, (2.30) N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide and (2.31) N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide.

(3) inhibitors of the respiratory chain at complex III, for example (3.1) ametoctradin (865318-97-4), (3.2) amisulbrom (348635-87-0), (3.3) azoxystrobin (131860-33-8), (3.4) cyazofamid (120116-88-3), (3.5) coumethoxystrobin (850881-30-0), (3.6) coumoxystrobin (850881-70-8), (3.7) dimoxystrobin (141600-52-4), (3.8) enestroburin (238410-11-2) (WO 2004/058723), (3.9) famoxadone (131807-57-3) (WO 2004/058723), (3.10) fenamidone (161326-34-7) (WO 2004/058723), (3.11) fenoxystrobin (918162-02-4), (3.12) fluoxastrobin (361377-29-9) (WO 2004/058723), (3.13) kresoxim-methyl (143390-89-0) (WO 2004/058723), (3.14) metominostrobin (133408-50-1) (WO 2004/058723), (3.15) orysastrobin (189892-69-1) (WO 2004/058723), (3.16) picoxystrobin (117428-22-5) (WO 2004/058723), (3.17) pyraclostrobin (175013-18-0) (WO 2004/058723), (3.18) pyrametostrobin (915410-70-7) (WO 2004/058723), (3.19) pyraoxystrobin (862588-11-2) (WO 2004/058723), (3.20) pyribencarb (799247-52-2) (WO 2004/058723), (3.21) triclopyricarb (902760-40-1), (3.22) trifloxystrobin (141517-21-7) (WO 2004/058723), (3.23) (2E)-2-(2-{[6-(3-chloro-2-methylphenoxy)-5-fluoropyrimidin-4-yl]oxy}phenyl)-2-(methoxyimino)-N-methylethanamide (WO 2004/058723), (3.24) (2E)-2-(methoxyimino)-N-methyl-2-(2-{[({(1E)-1-[3-(trifluoromethyl)phenyl]ethylidene}amino)oxy]methyl}phenyl)ethanamide (WO 2004/058723), (3.25) (2E)-2-(methoxyimino)-N-methyl-2-{2-[(E)-({1-[3-(trifluoromethyl)phenyl]ethoxy}imino)methyl]phenyl}ethanamide (158169-73-4), (3.26) (2E)-2-{2-[({[(1E)-1-(3-{[(E)-1-fluoro-2-phenylethenyl]oxy}phenyl)ethylidene]amino}oxy)methyl]phenyl}-2-(methoxyimino)-N-methylethanamide (326896-28-0), (3.27) (2E)-2-{2-[({[(2E,3E)-4-(2,6-dichlorophenyl)but-3-en-2-ylidene]amino}oxy)methyl]phenyl}-2-(methoxyimino)-N-methylethanamide, (3.28) 2-chloro-N-(1,1,3-trimethyl-2,3-dihydro-1H-inden-4-yl)pyridine-3-carboxamide (119899-14-8), (3.29) 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, (3.30) methyl (2E)-2-{2-[({cyclopropyl[(4-methoxyphenyl)imino]methyl}sulfanyl)methyl]phenyl}-3-methoxyprop-2-enoate (149601-03-6), (3.31) N-(3-ethyl-3,5,5-trimethylcyclohexyl)-3-(formylamino)-2-hydroxybenzamide (226551-21-9), (3.32) 2-{2-[(2,5-dimethylphenoxy)methyl]phenyl}-2-methoxy-N-methylacetamide (173662-97-0) and (3.33) (2R)-2-{2-[(2,5-dimethylphenoxy)methyl]phenyl}-2-methoxy-N-methylacetamide (394657-24-0).

(4) Inhibitors of the mitosis and cell division, for example (4.1) benomyl (17804-35-2), (4.2) carbendazim (10605-21-7), (4.3) chlorfenazole (3574-96-7), (4.4) diethofencarb (87130-20-9), (4.5) ethaboxam (162650-77-3), (4.6) fluopicolide (239110-15-7), (4.7) fuberidazole (3878-19-1), (4.8) pencycuron (66063-05-6), (4.9) thiabendazole (148-79-8), (4.10) thiophanate-methyl (23564-05-8), (4.11) thiophanate (23564-06-9), (4.12) zoxamide (156052-68-5), (4.13) 5-chloro-7-(4-methylpiperidin-1-yl)-6-(2,4,6-trifluorophenyl)[1,2,4]triazolo[1,5-a]pyrimidine (214706-53-3) and (4.14) 3-chloro-5-(6-chloropyridin-3-yl)-6-methyl-4-(2,4,6-trifluorophenyl)pyridazine (1002756-87-7).

(5) Compounds capable to have a multisite action, like for example (5.1) bordeaux mixture (8011-63-0), (5.2) captafol (2425-06-1), (5.3) captan (133-06-2) (WO 02/12172), (5.4) chlorothalonil (1897-45-6), (5.5) copper hydroxide (20427-59-2), (5.6) copper naphthenate (1338-02-9), (5.7) copper oxide (1317-39-1), (5.8) copper oxychloride (1332-40-7), (5.9) copper(2+) sulfate (7758-98-7), (5.10) dichlofluanid (1085-98-9), (5.11) dithianon (3347-22-6), (5.12) dodine (2439-10-3), (5.13) dodine free base, (5.14) ferbam (14484-64-1), (5.15) fluorofolpet (719-96-0), (5.16) folpet (133-07-3), (5.17) guazatine (108173-90-6), (5.18) guazatine acetate, (5.19) iminoctadine (13516-27-3), (5.20) iminoctadine albesilate (169202-06-6), (5.21) iminoctadine triacetate (57520-17-9), (5.22) mancopper (53988-93-5), (5.23) mancozeb (8018-01-7), (5.24) maneb (12427-38-2), (5.25) metiram (9006-42-2), (5.26) metiram zinc (9006-42-2), (5.27) oxine-copper (10380-28-6), (5.28) propamidine (104-32-5), (5.29) propineb (12071-83-9), (5.30) sulfur and sulfur preparations including calcium polysulfide (7704-34-9), (5.31) thiram (137-26-8), (5.32) tolylfluanid (731-27-1), (5.33) zineb (12122-67-7) and (5.34) ziram (137-30-4).

(6) Compounds capable to induce a host defence, for example (6.1) acibenzolar-5-methyl (135158-54-2), (6.2) isotianil (224049-04-1), (6.3) probenazole (27605-76-1) and (6.4) tiadinil (223580-51-6).

(7) Inhibitors of the amino acid and/or protein biosynthesis, for example (7.1) andoprim (23951-85-1), (7.2) blasticidin-S (2079-00-7), (7.3) cyprodinil (121552-61-2), (7.4) kasugamycin (6980-18-3), (7.5) kasugamycin hydrochloride hydrate (19408-46-9), (7.6) mepanipyrim (110235-47-7), (7.7) pyrimethanil (53112-28-0) and (7.8) 3-(5-fluoro-3,3,4,4-tetramethyl-3,4-dihydroisoquinolin-1-yl)quinoline (861647-32-7) (WO2005070917).

(8) Inhibitors of the ATP production, for example (8.1) fentin acetate (900-95-8), (8.2) fentin chloride (639-58-7), (8.3) fentin hydroxide (76-87-9) and (8.4) silthiofam (175217-20-6).

(9) Inhibitors of the cell wall synthesis, for example (9.1) benthiavalicarb (177406-68-7), (9.2) dimethomorph (110488-70-5), (9.3) flumorph (211867-47-9), (9.4) iprovalicarb (140923-17-7), (9.5) mandipropamid (374726-62-2), (9.6) polyoxins (11113-80-7), (9.7) polyoxorim (22976-86-9), (9.8) validamycin A (37248-47-8) and (9.9) valifenalate (283159-94-4; 283159-90-0).

(10) Inhibitors of the lipid and membrane synthesis, for example (10.1) biphenyl (92-52-4), (10.2) chloroneb (2675-77-6), (10.3) dicloran (99-30-9), (10.4) edifenphos (17109-49-8), (10.5) etridiazole (2593-15-9), (10.6) iodocarb (55406-53-6), (10.7) iprobenfos (26087-47-8), (10.8) isoprothiolane (50512-35-1), (10.9) propamocarb (25606-41-1), (10.10) propamocarb hydrochloride (25606-41-1), (10.11) prothiocarb (19622-08-3), (10.12) pyrazophos (13457-18-6), (10.13) quintozene (82-68-8), (10.14) tecnazene (117-18-0) and (10.15) tolclofos-methyl (57018-04-9).

(11) Inhibitors of the melanine biosynthesis, for example (11.1) carpropamid (104030-54-8), (11.2) diclocymet (139920-32-4), (11.3) fenoxanil (115852-48-7), (11.4) phthalide (27355-22-2), (11.5) pyroquilon (57369-32-1), (11.6) tricyclazole (41814-78-2) and (11.7) 2,2,2-trifluoroethyl {3-methyl-1-[(4-methylbenzoyl)amino]butan-2-yl}carbamate (851524-22-6) (WO2005042474).

(12) Inhibitors of the nucleic acid synthesis, for example (12.1) benalaxyl (71626-11-4), (12.2) benalaxyl-M (kiralaxyl) (98243-83-5), (12.3) bupirimate (41483-43-6), (12.4) clozylacon (67932-85-8), (12.5) dimethirimol (5221-53-4), (12.6) ethirimol (23947-60-6), (12.7) furalaxyl (57646-30-7), (12.8) hymexazol (10004-44-1), (12.9) metalaxyl (57837-19-1), (12.10) metalaxyl-M (mefenoxam) (70630-17-0), (12.11) ofurace (58810-48-3), (12.12) oxadixyl (77732-09-3) and (12.13) oxolinic acid (14698-29-4).

(13) Inhibitors of the signal transduction, for example (13.1) chlozolinate (84332-86-5), (13.2) fenpiclonil (74738-17-3), (13.3) fludioxonil (131341-86-1), (13.4) iprodione (36734-19-7), (13.5) procymidone (32809-16-8), (13.6) quinoxyfen (124495-18-7) and (13.7) vinclozolin (50471-44-8).

(14) Compounds capable to act as an uncoupler, for example (14.1) binapacryl (485-31-4), (14.2) dinocap (131-72-6), (14.3) ferimzone (89269-64-7), (14.4) fluazinam (79622-59-6) and (14.5) meptyldinocap (131-72-6).

(15) Further compounds, for example (15.1) benthiazole (21564-17-0), (15.2) bethoxazin (163269-30-5), (15.3) capsimycin (70694-08-5), (15.4) carvone (99-49-0), (15.5) chinomethionat (2439-01-2), (15.6) pyriofenone (chlazafenone) (688046-61-9), (15.7) cufraneb (11096-18-7), (15.8) cyflufenamid (180409-60-3), (15.9) cymoxanil (57966-95-7), (15.10) cyprosulfamide (221667-31-8), (15.11) dazomet (533-74-4), (15.12) debacarb (62732-91-6), (15.13) dichlorophen (97-23-4), (15.14) diclomezine (62865-36-5), (15.15) difenzoquat (49866-87-7), (15.16) difenzoquat methylsulfate (43222-48-6), (15.17) diphenylamine (122-39-4), (15.18) ecomate, (15.19) fenpyrazamine (473798-59-3), (15.20) flumetover (154025-04-4), (15.21) fluoroimide (41205-21-4), (15.22) flusulfamide (106917-52-6), (15.23) flutianil (304900-25-2), (15.24) fosetyl-aluminium (39148-24-8), (15.25) fosetyl-calcium, (15.26) fosetyl-sodium (39148-16-8), (15.27) hexachlorobenzene (118-74-1), (15.28) irumamycin (81604-73-1), (15.29) methasulfocarb (66952-49-6), (15.30) methyl isothiocyanate (556-61-6), (15.31) metrafenone (220899-03-6), (15.32) mildiomycin (67527-71-3), (15.33) natamycin (7681-93-8), (15.34) nickel dimethyldithiocarbamate (15521-65-0), (15.35) nitrothal-isopropyl (10552-74-6), (15.36) octhilinone (26530-20-1), (15.37) oxamocarb (917242-12-7), (15.38) oxyfenthiin (34407-87-9), (15.39) pentachlorophenol and salts (87-86-5), (15.40) phenothrin, (15.41) phosphorous acid and its salts (13598-36-2), (15.42) propamocarb-fosetylate, (15.43) propanosine-sodium (88498-02-6), (15.44) proquinazid (189278-12-4), (15.45) pyrimorph (868390-90-3), (15.45e) (2E)-3-(4-tert-butylphenyl)-3-(2-chloropyridin-4-yl)-1-(morpholin-4-yl)prop-2-en-1-one (1231776-28-5), (15.45z) (2Z)-3-(4-tert-butylphenyl)-3-(2-chloropyridin-4-yl)-1-(morpholin-4-yl)prop-2-en-1-one (1231776-29-6), (15.46) pyrrolnitrine (1018-71-9) (EP-A 1 559 320), (15.47) tebufloquin (376645-78-2), (15.48) tecloftalam (76280-91-6), (15.49) tolnifanide (304911-98-6), (15.50) triazoxide (72459-58-6), (15.51) trichlamide (70193-21-4), (15.52) zarilamid (84527-51-5), (15.53) (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate (517875-34-2) (WO2003035617), (15.54) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone (1003319-79-6) (WO 2008013622), (15.55) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone (1003319-80-9) (WO 2008013622), (15.56) 1-(4-{4-[5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone (1003318-67-9) (WO 2008013622), (15.57) 1-(4-methoxyphenoxy)-3,3-dimethylbutan-2-yl 1H-imidazole-1-carboxylate (111227-17-9), (15.58) 2,3,5,6-tetrachloro-4-(methylsulfonyl)pyridine (13108-52-6), (15.59) 2,3-dibutyl-6-chlorothieno[2,3-d]pyrimidin-4(3H)-one (221451-58-7), (15.60) 2,6-dimethyl-1H,5H-[1,4]dithiino[2,3-c:5,6-c′]dipyrrole-1,3,5,7(2H,6H)-tetrone, (15.61) 2-[5-methyl-3-(trifluoromethyl)-1H-pyrazol-1-yl]-1-(4-{4-[(5R)-5-phenyl-4,5-dihydro-1,2-oxazol-3-yl]-1,3-thiazol-2-yl}piperidin-1-yl)ethanone (1003316-53-7) (WO 2008013622), (15.62) 2-[5-methyl-3-(trifluoromethyl)-1H-pyrazol-1-yl]-1-(4-{4-[(5S)-5-phenyl-4,5-dihydro-1,2-oxazol-3-yl]-1,3-thiazol-2-yl}piperidin-1-yl)ethanone (1003316-54-8) (WO 2008013622), (15.63) 2-[5-methyl-3-(trifluoromethyl)-1H-pyrazol-1-yl]-1-{4-[4-(5-phenyl-4,5-dihydro-1,2-oxazol-3-yl)-1,3-thiazol-2-yl]piperidin-1-yl}ethanone (1003316-51-5) (WO 2008013622), (15.64) 2-butoxy-6-iodo-3-propyl-4H-chromen-4-one, (15.65) 2-chloro-5-[2-chloro-1-(2,6-difluoro-4-methoxyphenyl)-4-methyl-1H-imidazol-5-yl]pyridine, (15.66) 2-phenylphenol and salts (90-43-7), (15.67) 3-(4,4,5-trifluoro-3,3-dimethyl-3,4-dihydroisoquinolin-1-yl)quinoline (861647-85-0) (WO2005070917), (15.68) 3,4,5-trichloropyridine-2,6-dicarbonitrile (17824-85-0), (15.69) 3-[5-(4-chlorophenyl)-2,3-dimethyl-1,2-oxazolidin-3-yl]pyridine, (15.70) 3-chloro-5-(4-chlorophenyl)-4-(2,6-difluorophenyl)-6-methylpyridazine, (15.71) 4-(4-chlorophenyl)-5-(2,6-difluorophenyl)-3,6-dimethylpyridazine, (15.72) 5-amino-1,3,4-thiadiazole-2-thiol, (15.73) 5-chloro-N′-phenyl-N′-(prop-2-yn-1-yl)thiophene-2-sulfonohydrazide (134-31-6), (15.74) 5-fluoro-2-[(4-fluorobenzyl)oxy]pyrimidin-4-amine (1174376-11-4) (WO2009094442), (15.75) 5-fluoro-2-[(4-methylbenzyl)oxy]pyrimidin-4-amine (1174376-25-0) (WO2009094442), (15.76) 5-methyl-6-octyl[1,2,4]triazolo[1,5-a]pyrimidin-7-amine, (15.77) ethyl (2Z)-3-amino-2-cyano-3-phenylprop-2-enoate, (15.78) N′-(4-{[3-(4-chlorobenzyl)-1,2,4-thiadiazol-5-yl]oxy}-2,5-dimethylphenyl)-N-ethyl-N-methylimidoformamide, (15.79) N-(4-chlorobenzyl)-3-[3-methoxy-4-(prop-2-yn-1-yloxy)phenyl]propanamide, (15.80) N-[(4-chlorophenyl)(cyano)methyl]-3-[3-methoxy-4-(prop-2-yn-1-yloxy)phenyl]propanamide, (15.81) N-[(5-bromo-3-chloropyridin-2-yl)methyl]-2,4-dichloropyridine-3-carboxamide, (15.82) N-[1-(5-bromo-3-chloropyridin-2-yl)ethyl]-2,4-dichloropyridine-3-carboxamide, (15.83) N-[1-(5-bromo-3-chloropyridin-2-yl)ethyl]-2-fluoro-4-iodopyridine-3-carboxamide, (15.84) N-{(E)-[(cyclopropylmethoxy)imino][6-(difluoromethoxy)-2,3-difluorophenyl]methyl}-2-phenylacetamide (221201-92-9), (15.85) N-{(Z)-[(cyclopropylmethoxy)imino][6-(difluoromethoxy)-2,3-difluorophenyl]methyl}-2-phenylacetamide (221201-92-9), (15.86) N′-{4-[(3-tert-butyl-4-cyano-1,2-thiazol-5-yl)oxy]-2-chloro-5-methylphenyl}-N-ethyl-N-methylimidoformamide, (15.87) N-methyl-2-(1-{[5-methyl-3-(trifluoromethyl)-1H-pyrazol-1-yl]acetyl}piperidin-4-yl)-N-(1,2,3,4-tetrahydronaphthalen-1-yl)-1,3-thiazole-4-carboxamide (922514-49-6) (WO 2007014290), (15.88) N-methyl-2-(1-{[5-methyl-3-(trifluoromethyl)-1H-pyrazol-1-yl]acetyl}piperidin-4-O—N-[(1R)-1,2,3,4-tetrahydronaphthalen-1-yl]-1,3-thiazole-4-carboxamide (922514-07-6) (WO 2007014290), (15.89) N-methyl-2-(1-{[5-methyl-3-(trifluoromethyl)-1H-pyrazol-1-yl]acetyl}piperidin-4-yl)-N-[(1S)-1,2,3,4-tetrahydronaphthalen-1-yl]-1,3-thiazole-4-carboxamide (922514-48-5) (WO 2007014290), (15.90) pentyl {6-[({[(1-methyl-1H-tetrazol-5-yl)(phenyl)methylidene]amino}oxy)methyl]pyridin-2-yl}carbamate, (15.91) phenazine-1-carboxylic acid, (15.92) quinolin-8-ol (134-31-6), (15.93) quinolin-8-ol sulfate (2:1) (134-31-6) and (15.94) tert-butyl {6-[({[(1-methyl-1H-tetrazol-5-yl)(phenyl)methylene]amino}oxy)methyl]pyridin-2-yl}carbamate.

(16) Further compounds, for example (16.1) 1-methyl-3-(trifluoromethyl)-N-[2′-(trifluoromethyl)biphenyl-2-yl]-1H-pyrazole-4-carboxamide, (16.2) N-(4′-chlorobiphenyl-2-yl)-3-(difluoromethyl)-1-methyl-1H-pyrazole-4-carboxamide, (16.3) N-(2′,4′-dichlorobiphenyl-2-yl)-3-(difluoromethyl)-1-methyl-1H-pyrazole-4-carboxamide, (16.4) 3-(difluoromethyl)-1-methyl-N-[4′-(trifluoromethyl)biphenyl-2-yl]-1H-pyrazole-4-carboxamide, (16.5) N-(2′,5′-difluorobiphenyl-2-yl)-1-methyl-3-(trifluoromethyl)-1H-pyrazole-4-carboxamide, (16.6) 3-(difluoromethyl)-1-methyl-N-[4′-(prop-1-yn-1-yl)biphenyl-2-yl]-1H-pyrazole-4-carboxamide (known from WO 2004/058723), (16.7) 5-fluoro-1,3-dimethyl-N-[4′-(prop-1-yn-1-yl)biphenyl-2-yl]-1H-pyrazole-4-carboxamide (known from WO 2004/058723), (16.8) 2-chloro-N-[4′-(prop-1-yn-1-yl)biphenyl-2-yl]pyridine-3-carboxamide (known from WO 2004/058723), (16.9) 3-(difluoromethyl)-N-[4′-(3,3-dimethylbut-1-yn-1-yl)biphenyl-2-yl]-1-methyl-1H-pyrazole-4-carboxamide (known from WO 2004/058723), (16.10) N-[4′-(3,3-dimethylbut-1-yn-1-yl)biphenyl-2-yl]-5-fluoro-1,3-dimethyl-1H-pyrazole-4-carboxamide (known from WO 2004/058723), (16.11) 3-(difluoromethyl)-N-(4′-ethynylbiphenyl-2-yl)-1-methyl-1H-pyrazole-4-carboxamide (known from WO 2004/058723), (16.12) N-(4′-ethynylbiphenyl-2-yl)-5-fluoro-1,3-dimethyl-1H-pyrazole-4-carboxamide (known from WO 2004/058723), (16.13) 2-chloro-N-(4′-ethynylbiphenyl-2-yl)pyridine-3-carboxamide (known from WO 2004/058723), (16.14) 2-chloro-N-[4′-(3,3-dimethylbut-1-yn-1-yl)biphenyl-2-yl]pyridine-3-carboxamide (known from WO 2004/058723), (16.15) 4-(difluoromethyl)-2-methyl-N-[4′-(trifluoromethyl)biphenyl-2-yl]-1,3-thiazole-5-carboxamide (known from WO 2004/058723), (16.16) 5-fluoro-N-[4′-(3-hydroxy-3-methylbut-1-yn-1-yl)biphenyl-2-yl]-1,3-dimethyl-1H-pyrazole-4-carboxamide (known from WO 2004/058723), (16.17) 2-chloro-N-[4′-(3-hydroxy-3-methylbut-1-yn-1-yl)biphenyl-2-yl]pyridine-3-carboxamide (known from WO 2004/058723), (16.18) 3-(difluoromethyl)-N-[4′-(3-methoxy-3-methylbut-1-yn-1-yl)biphenyl-2-yl]-1-methyl-1H-pyrazole-4-carboxamide (known from WO 2004/058723), (16.19) 5-fluoro-N-[4′-(3-methoxy-3-methylbut-1-yn-1-yl)biphenyl-2-yl]-1,3-dimethyl-1H-pyrazole-4-carboxamide (known from WO 2004/058723), (16.20) 2-chloro-N-[4′-(3-methoxy-3-methylbut-1-yn-1-yl)biphenyl-2-yl]pyridine-3-carboxamide (known from WO 2004/058723), (16.21) (5-bromo-2-methoxy-4-methylpyridin-3-yl)(2,3,4-trimethoxy-6-methylphenyl)methanone (known from EP-A 1 559 320), (16.22) N-[2-(4-{[3-(4-chlorophenyl)prop-2-yn-1-yl]oxy}-3-methoxyphenyl)ethyl]-N2-(methylsulfonyl)valinamide (220706-93-4), (16.23) 4-oxo-4-[(2-phenylethyl)amino]butanoic acid and (16.24) but-3-yn-1-yl {6-[({[(Z)-(1-methyl-1H-tetrazol-5-yl)(phenyl)methylene]amino}oxy)methyl]pyridin-2-yl}carbamate.

All named mixing partners of the classes (1) to (16) can, if their functional groups enable this, optionally form salts with suitable bases or acids.

The composition according to the invention comprising a mixture of a compound of formula (I) with a bactericide compound can also be particularly advantageous. Examples of suitable bactericide mixing partners can be selected in the following list: bronopol, dichlorophen, nitrapyrin, nickel dimethyldithiocarbamate, kasugamycin, octhilinone, furancarboxylic acid, oxytetracycline, probenazole, streptomycin, tecloftalam, copper sulfate and other copper preparations.

The compounds of formula (I) and the fungicide composition according to the invention can be used to curatively or preventively control the phytopathogenic fungi of plants or crops.

Thus, according to a further aspect of the invention, there is provided a method for curatively or preventively controlling the phytopathogenic fungi of plants or crops characterised in that a compound of formula (I) or a fungicide composition according to the invention is applied to the seed, the plant or to the fruit of the plant or to the soil wherein the plant is growing or wherein it is desired to grow.

The method of treatment according to the invention can also be useful to treat propagation material such as tubers or rhizomes, but also seeds, seedlings or seedlings pricking out and plants or plants pricking out. This method of treatment can also be useful to treat roots. The method of treatment according to the invention can also be useful to treat the overground parts of the plant such as trunks, stems or stalks, leaves, flowers and fruit of the concerned plant.

According to the invention all plants and plant parts can be treated. By plants is meant all plants and plant populations such as desirable and undesirable wild plants, cultivars and plant varieties (whether or not protectable by plant variety or plant breeder's rights). Cultivars and plant varieties can be plants obtained by conventional propagation and breeding methods which can be assisted or supplemented by one or more biotechnological methods such as by use of double haploids, protoplast fusion, random and directed mutagenesis, molecular or genetic markers or by bioengineering and genetic engineering methods. By plant parts is meant all above ground and below ground parts and organs of plants such as shoot, leaf, blossom and root, whereby for example leaves, needles, stems, branches, blossoms, fruiting bodies, fruits and seed as well as roots, corms and rhizomes are listed. Crops and vegetative and generative propagating material, for example cuttings, corms, rhizomes, runners and seeds also belong to plant parts.

Among the plants that can be protected by the method according to the invention, mention may be made of major field crops like corn, soybean, cotton, Brassica oilseeds such as Brassica napus (e.g. canola), Brassica rapa, B. juncea (e.g. mustard) and Brassica carinata, rice, wheat, sugarbeet, sugarcane, oats, rye, barley, millet, triticale, flax, vine and various fruits and vegetables of various botanical taxa such as Rosaceae sp. (for instance pip fruit such as apples and pears, but also stone fruit such as apricots, cherries, almonds and peaches, berry fruits such as strawberries), Ribesioidae sp., Juglandaceae sp., Betulaceae sp., Anacardiaceae sp., Fagaceae sp., Moraceae sp., Oleaceae sp., Actimidaceae sp., Lauraceae sp., Musaceae sp. (for instance banana trees and plantings), Rubiaceae sp. (for instance coffee), Theaceae sp., Sterculiceae sp., Rutaceae sp. (for instance lemons, oranges and grapefruit); Solanaceae sp. (for instance tomatoes, potatoes, peppers, eggplant), Liliaceae sp., Compositiae sp. (for instance lettuce, artichoke and chicory—including root chicory, endive or common chicory), Umbelliferae sp. (for instance carrot, parsley, celery and celeriac), Cucurbitaceae sp. (for instance cucumber—including pickling cucumber, squash, watermelon, gourds and melons), Alliaceae sp. (for instance onions and leek), Cruciferae sp. (for instance white cabbage, red cabbage, broccoli, cauliflower, brussel sprouts, pak Choi, kohlrabi, radish, horseradish, cress, Chinese cabbage), Leguminosae sp. (for instance peanuts, peas and beans beans—such as climbing beans and broad beans), Chenopodiaceae sp. (for instance mangold, spinach beet, spinach, beetroots), Malvaceae (for instance okra), Asparagaceae (for instance asparagus); horticultural and forest crops; ornamental plants; as well as genetically modified homologues of these crops.

The method of treatment according to the invention can be used in the treatment of genetically modified organisms (GMOs), e.g. plants or seeds. Genetically modified plants (or transgenic plants) are plants of which a heterologous gene has been stably integrated into genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example, antisense technology, cosuppression technology or RNA interference—RNAi—technology). A heterologous gene that is located in the genome is also called a transgene. A transgene that is defined by its particular location in the plant genome is called a transformation or transgenic event.

Depending on the plant species or plant cultivars, their location and growth conditions (soils, climate, vegetation period, diet), the treatment according to the invention may also result in additional effects. Thus, for example, reduced application rates and/or a widening of the activity spectrum and/or an increase in the activity of the active compounds and compositions which can be used according to the invention, better plant growth, increased tolerance to high or low temperatures, increased tolerance to drought or to water or soil salt content, increased flowering performance, easier harvesting, accelerated maturation, higher harvest yields, bigger fruits, larger plant height, greener leaf color, earlier flowering, higher quality and/or a higher nutritional value of the harvested products, higher sugar concentration within the fruits, better storage stability and/or processability of the harvested products are possible, which exceed the effects which were actually to be expected.

At certain application rates, the active compound combinations according to the invention may also have a strengthening effect in plants. Accordingly, they are also suitable for mobilizing the defense system of the plant against attack by unwanted microorganisms. This may, if appropriate, be one of the reasons of the enhanced activity of the combinations according to the invention, for example against fungi. Plant-strengthening (resistance-inducing) substances are to be understood as meaning, in the present context, those substances or combinations of substances which are capable of stimulating the defense system of plants in such a way that, when subsequently inoculated with unwanted microorganisms, the treated plants display a substantial degree of resistance to these microorganisms. In the present case, unwanted microorganisms are to be understood as meaning phytopathogenic fungi, bacteria and viruses. Thus, the substances according to the invention can be employed for protecting plants against attack by the abovementioned pathogens within a certain period of time after the treatment. The period of time within which protection is effected generally extends from 1 to 10 days, preferably 1 to 7 days, after the treatment of the plants with the active compounds.

Plants and plant cultivars which are preferably to be treated according to the invention include all plants which have genetic material which impart particularly advantageous, useful traits to these plants (whether obtained by breeding and/or biotechnological means).

Plants and plant cultivars which are also preferably to be treated according to the invention are resistant against one or more biotic stresses, i.e. said plants show a better defense against animal and microbial pests, such as against nematodes, insects, mites, phytopathogenic fungi, bacteria, viruses and/or viroids.

Examples of nematode resistant plants are described in e.g. U.S. patent application Ser. Nos. 11/765,491, 11/765,494, 10/926,819, 10/782,020, 12/032,479, 10/783,417, 10/782,096, 11/657,964, 12/192,904, 11/396,808, 12/166,253, 12/166,239, 12/166,124, 12/166,209, 11/762,886, 12/364,335, 11/763,947, 12/252,453, 12/209,354, 12/491,396 or 12/497,221.

Plants and plant cultivars which may also be treated according to the invention are those plants which are resistant to one or more abiotic stresses. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients, shade avoidance.

Plants and plant cultivars which may also be treated according to the invention, are those plants characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including but not limited to, early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability.

Examples of plants with the above-mentioned traits are non-exhaustively listed in Table A.

Plants that may be treated according to the invention are hybrid plants that already express the characteristic of heterosis or hybrid vigor which results in generally higher yield, vigor, health and resistance towards biotic and abiotic stresses). Such plants are typically made by crossing an inbred male-sterile parent line (the female parent) with another inbred male-fertile parent line (the male parent). Hybrid seed is typically harvested from the male sterile plants and sold to growers. Male sterile plants can sometimes (e.g. in corn) be produced by detasseling, i.e. the mechanical removal of the male reproductive organs (or males flowers) but, more typically, male sterility is the result of genetic determinants in the plant genome. In that case, and especially when seed is the desired product to be harvested from the hybrid plants it is typically useful to ensure that male fertility in the hybrid plants is fully restored. This can be accomplished by ensuring that the male parents have appropriate fertility restorer genes which are capable of restoring the male fertility in hybrid plants that contain the genetic determinants responsible for male-sterility. Genetic determinants for male sterility may be located in the cytoplasm. Examples of cytoplasmic male sterility (CMS) were for instance described in Brassica species (WO 92/05251, WO 95/09910, WO 98/27806, WO 05/002324, WO 06/021972 and U.S. Pat. No. 6,229,072). However, genetic determinants for male sterility can also be located in the nuclear genome. Male sterile plants can also be obtained by plant biotechnology methods such as genetic engineering. A particularly useful means of obtaining male-sterile plants is described in WO 89/10396 in which, for example, a ribonuclease such as barnase is selectively expressed in the tapetum cells in the stamens. Fertility can then be restored by expression in the tapetum cells of a ribonuclease inhibitor such as barstar (e.g. WO 91/02069).

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-resistant plants are for example glyphosate-tolerant plants, i.e. plants made tolerant to the herbicide glyphosate or salts thereof. Plants can be made tolerant to glyphosate through different means. For example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium (Comai et al., 1983, Science 221, 370-371), the CP4 gene of the bacterium Agrobacterium sp. (Barry et al., 1992, Curr. Topics Plant Physiol. 7, 139-145), the genes encoding a Petunia EPSPS (Shah et al., 1986, Science 233, 478-481), a Tomato EPSPS (Gasser et al., 1988, J. Biol. Chem. 263, 4280-4289), or an Eleusine EPSPS (WO 01/66704). It can also be a mutated EPSPS as described in for example EP 0837944, WO 00/66746, WO 00/66747 or WO02/26995. Glyphosate-tolerant plants can also be obtained by expressing a gene that encodes a glyphosate oxido-reductase enzyme as described in U.S. Pat. Nos. 5,776,760 and 5,463,175. Glyphosate-tolerant plants can also be obtained by expressing a gene that encodes a glyphosate acetyl transferase enzyme as described in for example WO 02/36782, WO 03/092360, WO 05/012515 and WO 07/024,782. Glyphosate-tolerant plants can also be obtained by selecting plants containing naturally-occurring mutations of the above-mentioned genes, as described in for example WO 01/024615 or WO 03/013226. Plants expressing EPSPS genes that confer glyphosate tolerance are described in e.g. U.S. patent application Ser. Nos. 11/517,991, 10/739,610, 12/139,408, 12/352,532, 11/312,866, 11/315,678, 12/421,292, 11/400,598, 11/651,752, 11/681,285, 11/605,824, 12/468,205, 11/760,570, 11/762,526, 11/769,327, 11/769,255, 11/943,801 or 12/362,774. Plants comprising other genes that confer glyphosate tolerance, such as decarboxylase genes, are described in e.g. U.S. patent application Ser. Nos. 11/588,811, 11/185,342, 12/364,724, 11/185,560 or 12/423,926.

Other herbicide resistant plants are for example plants that are made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition, e.g. described in U.S. patent application Ser. No. 11/760,602. One such efficient detoxifying enzyme is an enzyme encoding a phosphinothricin acetyltransferase (such as the bar or pat protein from Streptomyces species). Plants expressing an exogenous phosphinothricin acetyltransferase are for example described in U.S. Pat. Nos. 5,561,236; 5,648,477; 5,646,024; 5,273,894; 5,637,489; 5,276,268; 5,739,082; 5,908,810 and 7,112,665.

Further herbicide-tolerant plants are also plants that are made tolerant to the herbicides inhibiting the enzyme hydroxyphenylpyruvatedioxygenase (HPPD). Hydroxyphenylpyruvatedioxygenases are enzymes that catalyze the reaction in which para-hydroxyphenylpyruvate (HPP) is transformed into homogentisate. Plants tolerant to HPPD-inhibitors can be transformed with a gene encoding a naturally-occurring resistant HPPD enzyme, or a gene encoding a mutated or chimeric HPPD enzyme as described in WO 96/38567, WO 99/24585, WO 99/24586, WO 2009/144079, WO 2002/046387, or U.S. Pat. No. 6,768,044. Tolerance to HPPD-inhibitors can also be obtained by transforming plants with genes encoding certain enzymes enabling the formation of homogentisate despite the inhibition of the native HPPD enzyme by the HPPD-inhibitor. Such plants and genes are described in WO 99/34008 and WO 02/36787. Tolerance of plants to HPPD inhibitors can also be improved by transforming plants with a gene encoding an enzyme having prephenate deshydrogenase (PDH) activity in addition to a gene encoding an HPPD-tolerant enzyme, as described in WO 2004/024928. Further, plants can be made more tolerant to HPPD-inhibitor herbicides by adding into their genome a gene encoding an enzyme capable of metabolizing or degrading HPPD inhibitors, such as the CYP450 enzymes shown in WO 2007/103567 and WO 2008/150473.

Still further herbicide resistant plants are plants that are made tolerant to acetolactate synthase (ALS) inhibitors. Known ALS-inhibitors include, for example, sulfonylurea, imidazolinone, triazolopyrimidines, pryimidinyoxy(thio)benzoates, and/or sulfonylaminocarbonyltriazolinone herbicides. Different mutations in the ALS enzyme (also known as acetohydroxyacid synthase, AHAS) are known to confer tolerance to different herbicides and groups of herbicides, as described for example in Tranel and Wright (2002, Weed Science 50:700-712), but also, in U.S. Pat. Nos. 5,605,011, 5,378,824, 5,141,870, and 5,013,659. The production of sulfonylurea-tolerant plants and imidazolinone-tolerant plants is described in U.S. Pat. Nos. 5,605,011; 5,013,659; 5,141,870; 5,767,361; 5,731,180; 5,304,732; 4,761,373; 5,331,107; 5,928,937; and 5,378,824; and international publication WO 96/33270. Other imidazolinone-tolerant plants are also described in for example WO 2004/040012, WO 2004/106529, WO 2005/020673, WO 2005/093093, WO 2006/007373, WO 2006/015376, WO 2006/024351, and WO 2006/060634. Further sulfonylurea- and imidazolinone-tolerant plants are also described in for example WO 07/024,782 and U.S. Patent Application No. 61/288,958.

Other plants tolerant to imidazolinone and/or sulfonylurea can be obtained by induced mutagenesis, selection in cell cultures in the presence of the herbicide or mutation breeding as described for example for soybeans in U.S. Pat. No. 5,084,082, for rice in WO 97/41218, for sugar beet in U.S. Pat. No. 5,773,702 and WO 99/057965, for lettuce in U.S. Pat. No. 5,198,599, or for sunflower in WO 01/065922.

Plants or plant cultivars (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

An “insect-resistant transgenic plant”, as used herein, includes any plant containing at least one transgene comprising a coding sequence encoding:
1) an insecticidal crystal protein from Bacillus thuringiensis or an insecticidal portion thereof, such as the insecticidal crystal proteins listed by Crickmore et al. (1998, Microbiology and Molecular Biology Reviews, 62: 807-813), updated by Crickmore et al. (2005) at the Bacillus thuringiensis toxin nomenclature, online at: http://www.lifesci.sussex.ac.uk/Home/Neil_Crickmore/Bt/), or insecticidal portions thereof, e.g., proteins of the Cry protein classes Cry1Ab, Cry1Ac, Cry1B, Cry1C, Cry1D, Cry1F, Cry2Ab, Cry3Aa, or Cry3Bb or insecticidal portions thereof (e.g. EP 1999141 and WO 2007/107302), or such proteins encoded by synthetic genes as e.g. described in and U.S. patent application Ser. No. 12/249,016; or
2) a crystal protein from Bacillus thuringiensis or a portion thereof which is insecticidal in the presence of a second other crystal protein from Bacillus thuringiensis or a portion thereof, such as the binary toxin made up of the Cry34 and Cry35 crystal proteins (Moellenbeck et al. 2001, Nat. Biotechnol. 19: 668-72; Schnepf et al. 2006, Applied Environm. Microbiol. 71, 1765-1774) or the binary toxin made up of the Cry1A or Cry1F proteins and the Cry2Aa or Cry2Ab or Cry2Ae proteins (U.S. patent application Ser. No. 12/214,022 and EP 08010791.5); or
3) a hybrid insecticidal protein comprising parts of different insecticidal crystal proteins from Bacillus thuringiensis, such as a hybrid of the proteins of 1) above or a hybrid of the proteins of 2) above, e.g., the Cry1A.105 protein produced by corn event MON89034 (WO 2007/027777); or
4) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation, such as the Cry3Bb1 protein in corn events MON863 or MON88017, or the Cry3A protein in corn event MIR604; or
5) an insecticidal secreted protein from Bacillus thuringiensis or Bacillus cereus, or an insecticidal portion thereof, such as the vegetative insecticidal (VIP) proteins listed at: http://www.lifesci.sussex.ac.uk/home/Neil_Crickmore/Bt/vip.html, e.g., proteins from the VIP3Aa protein class; or
6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins (WO 94/21795); or
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of 5) to 7) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation (while still encoding an insecticidal protein), such as the VIP3Aa protein in cotton event COT102; or
9) a secreted protein from Bacillus thuringiensis or Bacillus cereus which is insecticidal in the presence of a crystal protein from Bacillus thuringiensis, such as the binary toxin made up of VIP3 and Cry1A or Cry1F (U.S. Patent Appl. No. 61/126,083 and 61/195,019), or the binary toxin made up of the VIP3 protein and the Cry2Aa or Cry2Ab or Cry2Ae proteins (U.S. patent application Ser. No. 12/214,022 and EP 08010791.5).
10) a protein of 9) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation (while still encoding an insecticidal protein)

Of course, an insect-resistant transgenic plant, as used herein, also includes any plant comprising a combination of genes encoding the proteins of any one of the above classes 1 to 10. In one embodiment, an insect-resistant plant contains more than one transgene encoding a protein of any one of the above classes 1 to 10, to expand the range of target insect species affected when using different proteins directed at different target insect species, or to delay insect resistance development to the plants by using different proteins insecticidal to the same target insect species but having a different mode of action, such as binding to different receptor binding sites in the insect.

An “insect-resistant transgenic plant”, as used herein, further includes any plant containing at least one transgene comprising a sequence producing upon expression a double-stranded RNA which upon ingestion by a plant insect pest inhibits the growth of this insect pest, as described e.g. in WO 2007/080126, WO 2006/129204, WO 2007/074405, WO 2007/080127 and WO 2007/035650.

Plants or plant cultivars (obtained by plant biotechnology methods such as genetic engineering) which may also be treated according to the invention are tolerant to abiotic stresses. Such plants can be obtained by genetic transformation, or by selection of plants containing a mutation imparting such stress resistance. Particularly useful stress tolerance plants include:
1) plants which contain a transgene capable of reducing the expression and/or the activity of poly(ADP-ribose) polymerase (PARP) gene in the plant cells or plants as described in WO 00/04173, WO/2006/045633, EP 04077984.5, or EP 06009836.5.
2) plants which contain a stress tolerance enhancing transgene capable of reducing the expression and/or the activity of the PARG encoding genes of the plants or plants cells, as described e.g. in WO 2004/090140.
3) plants which contain a stress tolerance enhancing transgene coding for a plant-functional enzyme of the nicotineamide adenine dinucleotide salvage synthesis pathway including nicotinamidase, nicotinate phosphoribosyltransferase, nicotinic acid mononucleotide adenyl transferase, nicotinamide adenine dinucleotide synthetase or nicotine amide phosphorybosyltransferase as described e.g. in EP 04077624.7, WO 2006/133827, PCT/EP07/002,433, EP 1999263, or WO 2007/107326.

Plants or plant cultivars (obtained by plant biotechnology methods such as genetic engineering) which may also be treated according to the invention show altered quantity, quality and/or storage-stability of the harvested product and/or altered properties of specific ingredients of the harvested product such as:
1) transgenic plants which synthesize a modified starch, which in its physical-chemical characteristics, in particular the amylose content or the amylose/amylopectin ratio, the degree of branching, the average chain length, the side chain distribution, the viscosity behaviour, the gelling strength, the starch grain size and/or the starch grain morphology, is changed in comparison with the synthesised starch in wild type plant cells or plants, so that this is better suited for special applications. Said transgenic plants synthesizing a modified starch are disclosed, for example, in EP 0571427, WO 95/04826, EP 0719338, WO 96/15248, WO 96/19581, WO 96/27674, WO 97/11188, WO 97/26362, WO 97/32985, WO 97/42328, WO 97/44472, WO 97/45545, WO 98/27212, WO 98/40503, WO99/58688, WO 99/58690, WO 99/58654, WO 00/08184, WO 00/08185, WO 00/08175, WO 00/28052, WO 00/77229, WO 01/12782, WO 01/12826, WO 02/101059, WO 03/071860, WO 2004/056999, WO 2005/030942, WO 2005/030941, WO 2005/095632, WO 2005/095617, WO 2005/095619, WO 2005/095618, WO 2005/123927, WO 2006/018319, WO 2006/103107, WO 2006/108702, WO 2007/009823, WO 00/22140, WO 2006/063862, WO 2006/072603, WO 02/034923, EP 06090134.5, EP 06090228.5, EP 06090227.7, EP 07090007.1, EP 07090009.7, WO 01/14569, WO 02/79410, WO 03/33540, WO 2004/078983, WO 01/19975, WO 95/26407, WO 96/34968, WO 98/20145, WO 99/12950, WO 99/66050, WO 99/53072, U.S. Pat. No. 6,734,341, WO 00/11192, WO 98/22604, WO 98/32326, WO 01/98509, WO 01/98509, WO 2005/002359, U.S. Pat. No. 5,824,790, U.S. Pat. No. 6,013,861, WO 94/04693, WO 94/09144, WO 94/11520, WO 95/35026, WO 97/20936
2) transgenic plants which synthesize non starch carbohydrate polymers or which synthesize non starch carbohydrate polymers with altered properties in comparison to wild type plants without genetic modification. Examples are plants producing polyfructose, especially of the inulin and levan-type, as disclosed in EP 0663956, WO 96/01904, WO 96/21023, WO 98/39460, and WO 99/24593, plants producing alpha-1,4-glucans as disclosed in WO 95/31553, US 2002031826, U.S. Pat. No. 6,284,479, U.S. Pat. No. 5,712,107, WO 97/47806, WO 97/47807, WO 97/47808 and WO 00/14249, plants producing alpha-1,6 branched alpha-1,4-glucans, as disclosed in WO 00/73422, plants producing alternan, as disclosed in e.g. WO 00/47727, WO 00/73422, EP 06077301.7, U.S. Pat. No. 5,908,975 and EP 0728213,
3) transgenic plants which produce hyaluronan, as for example disclosed in WO 2006/032538, WO 2007/039314, WO 2007/039315, WO 2007/039316, JP 2006304779, and WO 2005/012529.
4) transgenic plants or hybrid plants, such as onions with characteristics such as ‘high soluble solids content’, ‘low pungency’ (LP) and/or ‘long storage’ (LS), as described in U.S. patent application Ser. No. 12/020,360 and 61/054,026.

Plants or plant cultivars (that can be obtained by plant biotechnology methods such as genetic engineering) which may also be treated according to the invention are plants, such as cotton plants, with altered fiber characteristics. Such plants can be obtained by genetic transformation, or by selection of plants contain a mutation imparting such altered fiber characteristics and include:
a) Plants, such as cotton plants, containing an altered form of cellulose synthase genes as described in WO 98/00549
b) Plants, such as cotton plants, containing an altered form of rsw2 or rsw3 homologous nucleic acids as described in WO 2004/053219
c) Plants, such as cotton plants, with increased expression of sucrose phosphate synthase as described in WO 01/17333
d) Plants, such as cotton plants, with increased expression of sucrose synthase as described in WO 02/45485
e) Plants, such as cotton plants, wherein the timing of the plasmodesmatal gating at the basis of the fiber cell is altered, e.g. through downregulation of fiber-selective β-1,3-glucanase as described in WO 2005/017157, or as described in EP 08075514.3 or U.S. Patent Appl. No. 61/128,938
f) Plants, such as cotton plants, having fibers with altered reactivity, e.g. through the expression of N-acetylglucosaminetransferase gene including nodC and chitin synthase genes as described in WO 2006/136351

Plants or plant cultivars (that can be obtained by plant biotechnology methods such as genetic engineering) which may also be treated according to the invention are plants, such as oilseed rape or related Brassica plants, with altered oil profile characteristics. Such plants can be obtained by genetic transformation, or by selection of plants contain a mutation imparting such altered oil profile characteristics and include:
a) Plants, such as oilseed rape plants, producing oil having a high oleic acid content as described e.g. in U.S. Pat. No. 5,969,169, U.S. Pat. No. 5,840,946 or U.S. Pat. No. 6,323,392 or U.S. Pat. No. 6,063,947
b) Plants such as oilseed rape plants, producing oil having a low linolenic acid content as described in U.S. Pat. No. 6,270,828, U.S. Pat. No. 6,169,190, or U.S. Pat. No. 5,965,755
c) Plant such as oilseed rape plants, producing oil having a low level of saturated fatty acids as described e.g. in U.S. Pat. No. 5,434,283 or U.S. patent application Ser. No. 12/668,303

Plants or plant cultivars (that can be obtained by plant biotechnology methods such as genetic engineering) which may also be treated according to the invention are plants, such as oilseed rape or related Brassica plants, with altered seed shattering characteristics. Such plants can be obtained by genetic transformation, or by selection of plants contain a mutation imparting such altered seed shattering characteristics and include plants such as oilseed rape plants with delayed or reduced seed shattering as described in U.S. Patent Appl. No. 61/135,230 WO09/068,313 and WO10/006,732.

Particularly useful transgenic plants which may be treated according to the invention are plants containing transformation events, or combination of transformation events, that are the subject of petitions for non-regulated status, in the United States of America, to the Animal and Plant Health Inspection Service (APHIS) of the United States Department of Agriculture (USDA) whether such petitions are granted or are still pending. At any time this information is readily available from APHIS (4700 River Road Riverdale, Md. 20737, USA), for instance on its internet site (URL http://www.aphis.usda.gov/brs/notreg.html). On the filing date of this application the petitions for nonregulated status that were pending with APHIS or granted by APHIS were those listed in table B which contains the following information:
Petition: the identification number of the petition. Technical descriptions of the transformation events can be found in the individual petition documents which are obtainable from APHIS, for example on the APHIS website, by reference to this petition number. These descriptions are herein incorporated by reference.
Extension of Petition: reference to a previous petition for which an extension is requested.
Institution: the name of the entity submitting the petition.
Regulated article: the plant species concerned.
Transgenic phenotype: the trait conferred to the plants by the transformation event.
Transformation event or line: the name of the event or events (sometimes also designated as lines or lines) for which nonregulated status is requested.
APHIS documents: various documents published by APHIS in relation to the Petition and which can be requested with APHIS.

Additional particularly useful plants containing single transformation events or combinations of transformation events are listed for example in the databases from various national or regional regulatory agencies (see for example http://gmoinfo.jrc.it/gmp_browse.aspx and http://www.agbios.com/dbase.php).

Further particularly transgenic plants include plants containing a transgene in an agronomically neutral or beneficial position as described in any of the patent publications listed in Table C.

TABLE A

| Trait | Reference | |
|---|---|---|
| Water use efficiency | WO 2000/073475 | |
| Nitrogen use efficiency | WO 1995/009911 | WO 2007/076115 |
| | WO 1997/030163 | WO 2005/103270 |
| | WO 2007/092704 | WO 2002/002776 |
| Improved photosynthesis | WO 2008/056915 | WO 2004/101751 |
| Nematode resistance | WO 1995/020669 | WO 2003/033651 |
| | WO 2001/051627 | WO 1999/060141 |
| | WO 2008/139334 | WO 1998/012335 |
| | WO 2008/095972 | WO 1996/030517 |
| | WO 2006/085966 | WO 1993/018170 |
| Reduced pod dehiscence | WO 2006/009649 | WO 1997/013865 |
| | WO 2004/113542 | WO 1996/030529 |
| | WO 1999/015680 | WO 1994/023043 |
| | WO 1999/000502 | |
| Aphid resistance | WO 2006/125065 | WO 2008/067043 |
| | WO 1997/046080 | WO 2004/072109 |
| Sclerotinia resistance | WO 2006/135717 | WO 2005/000007 |
| | WO 2006/055851 | WO 2002/099385 |
| | WO 2005/090578 | WO 2002/061043 |
| Botrytis resistance | WO 2006/046861 | WO 2002/085105 |
| Bremia resistance | US 20070022496 | WO 2004/049786 |
| | WO 2000/063432 | |
| Erwinia resistance | WO 2004/049786 | |
| Closterovirus resistance | WO 2007/073167 | WO 2002/022836 |
| | WO 2007/053015 | |
| Stress tolerance (including | WO 2010/019838 | WO2008/002480 |
| drought tolerance) | WO 2009/049110 | WO2005/033318 |
| Tobamovirus resistance | WO 2006/038794 | |

TABLE B
Petitions of Nonregulated Status Granted or Pending by APHIS as of Mar. 31, 2010

| Applicant Documents | | | | | |
|---|---|---|---|---|---|
| | Extension of | | | | |
| | Petition | | Regulated | Transgenic | Transformation |
| Petition | Number *** | Institution | Article | Phenotype | Event or Line |

Petitions for Nonregulated Status Pending

| | | | | |
|---|---|---|---|---|
| 10-070-01p | Virginia Tech | Peanut | Sclerotinia blight | N70, P39, and |
| | | | resistant | W171 |
| 09-349-01p | Dow | Soybean | Herbicide Tolerant | DAS-68416-4 |
| | AgroSciences | | | |
| 09-328-01p | Bayer Crop | Soybean | Herbicide Tolerant | FG72 |
| | Science | | | |
| 09-233-01p | Dow | Corn | Herbicide Tolerant | DAS-40278-9 |
| 09-201-01p | Monsanto | Soybean | | MON-877Ø5-6 |
| 09-183-01p | Monsanto | Soybean | | MON-87769 |
| 09-082-01p | Monsanto | Soybean | Lepidopteran resistant | MON 87701 |
| 09-063-01p | Stine Seed | Corn | Glyphosate tolerant | HCEM485 |
| 09-055-01p | Monsanto | Corn | Drought Tolerant | MON 87460 |
| 09-015-01p | BASF Plant | Soybean | Herbicide Tolerant | BPS-CV127-9 |
| | Science, LLC | | | Soybean |
| 08-366-01p | ArborGen | Eucalyptus | Freeze Tolerant, | ARB-FTE1-08 |
| | | | Fertility Altered | |
| 08-340-01p | Bayer | Cotton | Glufosinate Tolerant, | T304-40XGHB119 |
| | | | Insect Resistant | |
| 08-338-01p | Pioneer | Corn | Male Sterile, Fertility | DP-32138-1 |
| | | | Restored, Visual | |
| | | | Marker | |
| 08-315-01p | Florigene | Rose | Altered Flower Color | IFD-524Ø1-4 and |
| | | | | IFD-529Ø1-9 |
| 07-253-01p | Syngenta | Corn | Lepidopteran resistant | MIR-162 Maize |
| 07-108-01p | Syngenta | Cotton | Lepidopteran Resistant | COT67B |
| 06-354-01p | Pioneer | Soybean | High Oleic Acid | DP-3Ø5423-1 |
| 05-280-01p | Syngenta | Corn | Thermostable alpha- | 3272 |
| | | | amylase | |
| 04-110-01p | Monsanto & | Alfalfa | Glyphosate Tolerant | J101, J163 |
| | Forage Genetics | | | |
| 03-104-01p | Monsanto & | Creeping | Glyphosate Tolerant | ASR368 |
| | Scotts | bentgrass | | |

Petitions for Nonregulated Status Granted

| | | | | | |
|---|---|---|---|---|---|
| 07-152-01p | | Pioneer | Corn | glyphosate & | DP-098140-6 |
| | | | | Imidazolinone tolerant | |
| 04-337-01p | | University of | Papaya | Papaya Ringspot Virus | X17-2 |
| | | Florida | | Resistant | |
| 06-332-01p | | Bayer | Cotton | Glyphosate tolerant | GHB614 |
| | | CropScience | | | |
| 06-298-01p | | Monsanto | Corn | European Corn Borer | MON 89034 |
| | | | | resistant | |
| 06-271-01p | | Pioneer | Soybean | Glyphosate & | 356043 |
| | | | | acetolactate synthase | (DP-356Ø43-5) |
| | | | | tolerant | |
| 06-234-01p | 98-329-01p | Bayer | Rice | Phosphinothricin | LLRICE601 |
| | | CropScience | | tolerant | |
| 06-178-01p | | Monsanto | Soybean | Glyphosate tolerant | MON 89788 |
| 04-362-01p | | Syngenta | Corn | Corn Rootworm | MIR604 |
| | | | | Protected | |
| 04-264-01p | | ARS | Plum | Plum Pox Virus | C5 |
| | | | | Resistant | |
| 04-229-01p | | Monsanto | Corn | High Lysine | LY038 |
| 04-125-01p | | Monsanto | Corn | Corn Rootworm | 88017 |
| | | | | Resistant | |
| 04-086-01p | | Monsanto | Cotton | Glyphosate Tolerant | MON 88913 |
| 03-353-01p | | Dow | Corn | Corn Rootworm | 59122 |
| | | | | Resistant | |
| 03-323-01p | | Monsanto | Sugar | Glyphosate Tolerant | H7-1 |
| | | | Beet | | |
| 03-181-01p | 00-136-01p | Dow | Corn | Lepidopteran Resistant | TC-6275 |
| | | | | & Phosphinothricin | |
| | | | | tolerant | |
| 03-155-01p | | Syngenta | Cotton | Lepidopteran Resistant | COT 102 |
| 03-036-01p | | Mycogen/Dow | Cotton | Lepidopteran Resistant | 281-24-236 |
| 03-036-02p | | Mycogen/Dow | Cotton | Lepidopteran Resistant | 3006-210-23 |
| 02-042-01p | | Aventis | Cotton | Phosphinothericin | LLCotton25 |
| | | | | tolerant | |
| 01-324-01p | 98-216-01p | Monsanto | Rapeseed | Glyphosate tolerant | RT200 |
| 01-206-01p | 98-278-01p | Aventis | Rapeseed | Phosphinothricin | MS1 & RF1/RF2 |
| | | | | tolerant & pollination | |
| | | | | control | |
| 01-206-02p | 97-205-01p | Aventis | Rapeseed | Phosphinothricin | Topas 19/2 |
| | | | | tolerant | |
| 01-137-01p | | Monsanto | Corn | Corn Rootworm | MON 863 |
| | | | | Resistant | |
| 01-121-01p | | Vector | Tobacco | Reduced nicotine | Vector 21-41 |
| 00-342-01p | | Monsanto | Cotton | Lepidopteran resistant | Cotton Event |
| | | | | | 15985 |
| 00-136-01p | | Mycogen c/o | Corn | Lepidopteran resistant | Line 1507 |
| | | Dow & Pioneer | | phosphinothricin | |
| | | | | tolerant | |
| 00-011-01p | 97-099-01p | Monsanto | Corn | Glyphosate tolerant | NK603 |
| 99-173-01p | 97-204-01p | Monsanto | Potato | PLRV & CPB resistant | RBMT22-82 |
| 98-349-01p | 95-228-01p | AgrEvo | Corn | Phosphinothricin | MS6 |
| | | | | tolerant and Male | |
| | | | | sterile | |
| 98-335-01p | | U. of | Flax | Tolerant to soil | CDC Triffid |
| | | Saskatchewan | | residues of sulfonyl | |
| | | | | urea herbicide | |
| 98-329-01p | | AgrEvo | Rice | Phosphinothricin | LLRICE06, |
| | | | | tolerant | LLRICE62 |
| 98-278-01p | | AgrEvo | Rapeseed | Phosphinothricin | MS8 & RF3 |
| | | | | tolerant & Pollination | |
| | | | | control | |
| 98-238-01p | | AgrEvo | Soybean | Phosphinothricin | GU262 |
| | | | | tolerant | |
| 98-216-01p | | Monsanto | Rapeseed | Glyphosate tolerant | RT73 |
| 98-173-01p | | Novartis Seeds & | Beet | Glyphosate tolerant | GTSB77 |
| | | Monsanto | | | |
| 98-014-01p | 96-068-01p | AgrEvo | Soybean | Phosphinothricin | A5547-127 |
| | | | | tolerant | |
| 97-342-01p | | Pioneer | Corn | Male sterile & | 676, 678, 680 |
| | | | | Phosphinothricin | |
| | | | | tolerant | |
| 97-339-01p | | Monsanto | Potato | CPB & PVY resistant | RBMT15-101, |
| | | | | | SEMT15-02, |
| | | | | | SEMT15-15 |
| 97-336-01p | | AgrEvo | Beet | Phosphinothricin | T-120-7 |
| | | | | tolerant | |
| 97-287-01p | | Monsanto | Tomato | Lepidopteran resistant | 5345 |
| 97-265-01p | | AgrEvo | Corn | Phosphinothricin | CBH-351 |
| | | | | tolerant & Lep. | |
| | | | | resistant | |
| 97-205-01p | | AgrEvo | Rapeseed | Phosphinothricin | T45 |
| | | | | tolerant | |
| 97-204-01p | | Monsanto | Potato | CPB & PLRV resistant | RBMT21-129 & |
| | | | | | RBMT21-350 |
| 97-148-01p | | Bejo | Cichorium | Male sterile | RM3-3, RM3-4, |
| | | | intybus | | RM3-6 |
| 97-099-01p | | Monsanto | Corn | Glyphosate tolerant | GA21 |
| 97-013-01p | | Calgene | Cotton | Bromoxynil tolerant & | Events 31807 & |
| | | | | Lepidopteran resistant | 31808 |
| 97-008-01p | | Du Pont | Soybean | Oil profile altered | G94-1, G94-19, G- |
| | | | | | 168 |
| 96-317-01p | | Monsanto | Corn | Glyphosate tolerant & | MON802 |
| | | | | ECB resistant | |
| 96-291-01p | | DeKalb | Corn | European Corn Borer | DBT418 |
| | | | | resistant | |
| 96-248-01p | 92-196-01p | Calgene | Tomato | Fruit ripening altered | 1 additional |
| | | | | | FLAVRSAVR line |
| 96-068-01p | | AgrEvo | Soybean | Phosphinothricin | W62, W98, A2704- |
| | | | | tolerant | 12, A2704-21, |
| | | | | | A5547-35 |
| 96-051-01p | | Cornell U | Papaya | PRSV resistant | 55-1, 63-1 |
| 96-017-01p | 95-093-01p | Monsanto | Corn | European Corn Borer | MON809 & |
| | | | | resistant | MON810 |
| 95-352-01p | | Asgrow | Squash | CMV, ZYMV, WMV2 | CZW-3 |
| | | | | resistant | |
| 95-338-01p | | Monsanto | Potato | CPB resistant | SBT02-5 & -7, |
| | | | | | ATBT04-6 |
| | | | | | &-27, -30, -31, -36 |
| 95-324-01p | | Agritope | Tomato | Fruit ripening altered | 35 1 N |
| 95-256-01p | | Du Pont | Cotton | Sulfonylurea tolerant | 19-51a |
| 95-228-01p | | Plant Genetic | Corn | Male sterile | MS3 |
| | | Systems | | | |
| 95-195-01p | | Northrup King | Corn | European Corn Borer | Bt11 |
| | | | | resistant | |
| 95-179-01p | 92-196-01p | Calgene | Tomato | Fruit ripening altered | 2 additional |
| | | | | | FLAVRSAVR lines |
| 95-145-01p | | DeKalb | Corn | Phosphinothricin | B16 |
| | | | | tolerant | |
| 95-093-01p | | Monsanto | Corn | Lepidopteran resistant | MON 80100 |
| 95-053-01p | | Monsanto | Tomato | Fruit ripening altered | 8338 |
| 95-045-01p | | Monsanto | Cotton | Glyphosate tolerant | 1445, 1698 |
| 95-030-01p | 92-196-01p | Calgene | Tomato | Fruit ripening altered | 20 additional |
| | | | | | FLAVRSAVR lines |
| 94-357-01p | | AgrEvo | Corn | Phosphinothricin | T14, T25 |
| | | | | tolerant | |
| 94-319-01p | | Ciba Seeds | Corn | Lepidopteran resistant | Event 176 |
| 94-308-01p | | Monsanto | Cotton | Lepidopteran resistant | 531, 757, 1076 |
| 94-290-01p | | Zeneca & | Tomato | Fruit polygalacturonase | B, Da, F |
| | | Petoseed | | level decreased | |
| 94-257-01p | | Monsanto | Potato | Coleopteran resistant | BT6, BT10, BT12, |
| | | | | | BT16, BT17, BT18, |
| | | | | | BT23 |
| 94-230-01p | 92-196-01p | Calgene | Tomato | Fruit ripening altered | 9 additional |
| | | | | | FLAVRSAVR lines |
| 94-228-01p | | DNA Plant Tech | Tomato | Fruit ripening altered | 1345-4 |
| 94-227-01p | 92-196-01p | Calgene | Tomato | Fruit ripening altered | Line N73 1436-111 |
| 94-090-01p | | Calgene | Rapeseed | Oil profile altered | pCGN3828- |
| | | | | | 212/86- 18 & 23 |
| 93-258-01p | | Monsanto | Soybean | Glyphosate tolerant | 40-3-2 |
| 93-196-01p | | Calgene | Cotton | Bromoxynil tolerant | BXN |
| 92-204-01p | | Upjohn | Squash | WMV2 & ZYMV | ZW-20 |
| | | | | resistant | |
| 92-196-01p | | Calgene | Tomato | Fruit ripening altered | FLAVR SAVR |

NOTE:
To obtain the most up-to-date list of Crops No Longer Regulated, please look at the Current Status of Petitions. This list is automatically updated and reflects all petitions received to date by APHIS, including petitions pending, withdrawn, or approved.
Abbreviations:
CMV—cucumber mosaic virus; CPB—colorado potato beetle; PLRV—potato leafroll virus; PRSV—papaya ringspot virus; PVY—potato virus Y; WMV2—watermelon mosaic virus 2 ZYMV—zucchini yellow mosaic virus
*** Extension of Petition Number: Under 7CFR 340.6(e) a person may request that APHIS extend a determination of non-regulated status to other organisms based on their similarity of the previously deregulated article. This column lists the previously granted petition of that degregulated article.
**** Preliminary EA: The Environmental Assessment initially available for Public comment prior to finalization.

TABLE C

| Plant species | Event | Trait | Patent reference |
|---|---|---|---|
| Corn | PV-ZMGT32 (NK603) | Glyphosate tolerance | US 2007-056056 |
| Corn | MIR604 | Insect resistance (Cry3a055) | EP 1 737 290 |
| Corn | LY038 | High lysine content | U.S. Pat. No. 7,157,281 |
| Corn | 3272 | Self processing corn (alpha- | US 2006-230473 |
| | | amylase) | |
| Corn | PV-ZMIR13 | Insect resistance (Cry3Bb) | US 2006-095986 |
| | (MON863) | | |
| Corn | DAS-59122-7 | Insect resistance | US 2006-070139 |
| | | (Cry34Ab1/Cry35Ab1) | |
| Corn | TC1507 | Insect resistance (Cry1F) | U.S. Pat. No. 7,435,807 |
| Corn | MON810 | Insect resistance (Cry1Ab) | US 2004-180373 |
| Corn | VIP1034 | Insect resistance | WO 03/052073 |
| Corn | B16 | Glufosinate resistance | US 2003-126634 |
| Corn | GA21 | Glyphosate resistance | U.S. Pat. No. 6,040,497 |
| Corn | GG25 | Glyphosate resistance | U.S. Pat. No. 6,040,497 |
| Corn | GJ11 | Glyphosate resistance | U.S. Pat. No. 6,040,497 |
| Corn | FI117 | Glyphosate resistance | U.S. Pat. No. 6,040,497 |
| Corn | GAT-ZM1 | Glufosinate tolerance | WO 01/51654 |
| Corn | MON87460 | Drought tolerance | WO 2009/111263 |
| Corn | DP-098140-6 | Glyphosate tolerance/ALS | WO 2008/112019 |
| | | inhibitor tolerance | |
| Wheat | Event 1 | Fusarium resistance | CA 2561992 |
| | | (trichothecene 3-O- | |
| | | acetyltransferase) | |
| Sugar beet | T227-1 | Glyphosate tolerance | US 2004-117870 |
| Sugar beet | H7-1 | Glyphosate tolerance | WO 2004-074492 |
| Soybean | MON89788 | Glyphosate tolerance | US 2006-282915 |
| Soybean | A2704-12 | Glufosinate tolerance | WO 2006/108674 |
| Soybean | A5547-35 | Glufosinate tolerance | WO 2006/108675 |
| Soybean | DP-305423-1 | High oleic acid/ALS inhibitor | WO 2008/054747 |
| | | tolerance | |
| Rice | GAT-OS2 | Glufosinate tolerance | WO 01/83818 |
| Rice | GAT-OS3 | Glufosinate tolerance | US 2008-289060 |
| Rice | PE-7 | Insect resistance (Cry1Ac) | WO 2008/114282 |
| Oilseed rape | MS-B2 | Male sterility | WO 01/31042 |
| Oilseed rape | MS-BN1/RF-BN1 | Male sterility/restoration | WO 01/41558 |
| Oilseed rape | RT73 | Glyphosate resistance | WO 02/36831 |
| Cotton | CE43-67B | Insect resistance (Cry1Ab) | WO 2006/128573 |
| Cotton | CE46-02A | Insect resistance (Cry1Ab) | WO 2006/128572 |
| Cotton | CE44-69D | Insect resistance (Cry1Ab) | WO 2006/128571 |
| Cotton | 1143-14A | Insect resistance (Cry1Ab) | WO 2006/128569 |
| Cotton | 1143-51B | Insect resistance (Cry1Ab) | WO 2006/128570 |
| Cotton | T342-142 | Insect resistance (Cry1Ab) | WO 2006/128568 |
| Cotton | event3006-210-23 | Insect resistance (Cry1Ac) | WO 2005/103266 |
| Cotton | PV-GHGT07 (1445) | Glyphosate tolerance | US 2004-148666 |
| Cotton | MON88913 | Glyphosate tolerance | WO 2004/072235 |
| Cotton | EE-GH3 | Glyphosate tolerance | WO 2007/017186 |
| Cotton | T304-40 | Insect-resistance (Cry1Ab) | WO2008/122406 |
| Cotton | Cot202 | Insect resistance (VIP3) | US 2007-067868 |
| Cotton | LLcotton25 | Glufosinate resistance | WO 2007/017186 |
| Cotton | EE-GH5 | Insect resistance (Cry1Ab) | WO 2008/122406 |
| Cotton | event 281-24-236 | Insect resistance (Cry1F) | WO 2005/103266 |
| Cotton | Cot102 | Insect resistance (Vip3A) | US 2006-130175 |
| Cotton | MON 15985 | Insect resistance (Cry1A/Cry2Ab) | US 2004-250317 |
| Bent Grass | Asr-368 | Glyphosate tolerance | US 2006-162007 |
| Brinjal | EE-1 | Insect resistance (Cry1Ac) | WO 2007/091277 |

Among the diseases of plants or crops that can be controlled by the method according to the invention, mention can be made of:

Powdery mildew diseases such as:
Blumeria diseases, caused for example by Blumeria graminis;
Podosphaera diseases, caused for example by Podosphaera leucotricha;
Sphaerotheca diseases, caused for example by Sphaerotheca fuliginea;
Uncinula diseases, caused for example by Uncinula necator;

Rust diseases such as:
Gymnosporangium diseases, caused for example by Gymnosporangium sabinae;
Hemileia diseases, caused for example by Hemileia vastatrix;
Phakopsora diseases, caused for example by Phakopsora pachyrhizi or Phakopsora meibomiae;
Puccinia diseases, caused for example by Puccinia recondite, Puccinia graminis or Puccinia striiformis;
Uromyces diseases, caused for example by Uromyces appendiculatus;

Oomycete diseases such as:
Albugo diseases caused for example by Albugo candida;
Bremia diseases, caused for example by Bremia lactucae;
Peronospora diseases, caused for example by Peronospora pisi or P. brassicae;
Phytophthora diseases, caused for example by Phytophthora infestans;
Plasmopara diseases, caused for example by Plasmopara viticola;
Pseudoperonospora diseases, caused for example by Pseudoperonospora humuli or
Pseudoperonospora cubensis;
Pythium diseases, caused for example by Pythium ultimum;

Leafspot, leaf blotch and leaf blight diseases such as:
Alternaria diseases, caused for example by Alternaria solani;
Cercospora diseases, caused for example by Cercospora beticola;
Cladiosporum diseases, caused for example by Cladiosporium cucumerinum;
Cochliobolus diseases, caused for example by Cochliobolus sativus (Conidiaform: Drechslera, Syn: Helminthosporium) or Cochliobolus miyabeanus;
Colletotrichum diseases, caused for example by Colletotrichum lindemuthanium;
Cycloconium diseases, caused for example by Cycloconium oleaginum;
Diaporthe diseases, caused for example by Diaporthe citri;
Elsinoe diseases, caused for example by Elsinoe fawcettii;
Gloeosporium diseases, caused for example by Gloeosporium laeticolor;
Glomerella diseases, caused for example by Glomerella cingulata;
Guignardia diseases, caused for example by Guignardia bidwelli;
Leptosphaeria diseases, caused for example by Leptosphaeria maculans; Leptosphaeria nodorum;
Magnaporthe diseases, caused for example by Magnaporthe grisea;
Mycosphaerella diseases, caused for example by Mycosphaerella graminicola; Mycosphaerella arachidicola; Mycosphaerella fijiensis;
Phaeosphaeria diseases, caused for example by Phaeosphaeria nodorum;
Pyrenophora diseases, caused for example by Pyrenophora teres, or Pyrenophora tritici repentis;
Ramularia diseases, caused for example by Ramularia collo-cygni, or Ramularia areola;
Rhynchosporium diseases, caused for example by Rhynchosporium secalis;
Septoria diseases, caused for example by Septoria apii or Septoria lycopercisi;
Typhula diseases, caused for example by Typhula incarnate;
Venturia diseases, caused for example by Venturia inaequalis; Root, Sheath and stem diseases such as:
Corticium diseases, caused for example by Corticium graminearum;
Fusarium diseases, caused for example by Fusarium oxysporum;
Gaeumannomyces diseases, caused for example by Gaeumannomyces graminis;
Rhizoctonia diseases, caused for example by Rhizoctonia solani;
Sarocladium diseases caused for example by Sarocladium oryzae;
Sclerotium diseases caused for example by Sclerotium oryzae;
Tapesia diseases, caused for example by Tapesia acuformis;
Thielaviopsis diseases, caused for example by Thielaviopsis basicola;

Ear and panicle diseases such as:
Alternaria diseases, caused for example by Alternaria spp.;
Aspergillus diseases, caused for example by Aspergillus flavus;
Cladosporium diseases, caused for example by Cladosporium spp.;
Claviceps diseases, caused for example by Claviceps purpurea;
Fusarium diseases, caused for example by Fusarium culmorum;
Gibberella diseases, caused for example by Gibberella zeae;
Monographella diseases, caused for example by Monographella nivalis;

Smut and bunt diseases such as:
Sphacelotheca diseases, caused for example by Sphacelotheca reiliana;
Tilletia diseases, caused for example by Tilletia caries;
Urocystis diseases, caused for example by Urocystis occulta;
Ustilago diseases, caused for example by Ustilago nuda;

Fruit rot and mould diseases such as:
Aspergillus diseases, caused for example by Aspergillus flavus;
Botrytis diseases, caused for example by Botrytis cinerea;
Penicillium diseases, caused for example by Penicillium expansum;
Rhizopus diseases caused by example by Rhizopus stolonifer
Sclerotinia diseases, caused for example by Sclerotinia sclerotiorum;
Verticilium diseases, caused for example by Verticilium alboatrum;

Seed and soilborne decay, mould, wilt, rot and damping-off diseases:
Alternaria diseases, caused for example by Alternaria brassicicola
Aphanomyces diseases, caused for example by Aphanomyces euteiches
Ascochyta diseases, caused for example by Ascochyta lentis
Aspergillus diseases, caused for example by Aspergillus flavus
Cladosporium diseases, caused for example by Cladosporium herbarum
Cochliobolus diseases, caused for example by Cochliobolus sativus
(Conidiaform: Drechslera, Bipolaris Syn: Helminthosporium);
Colletotrichum diseases, caused for example by Colletotrichum coccodes;
Fusarium diseases, caused for example by Fusarium culmorum;
Gibberella diseases, caused for example by Gibberella zeae;
Macrophomina diseases, caused for example by Macrophomina phaseolina
Monographella diseases, caused for example by Monographella nivalis;
Penicillium diseases, caused for example by Penicillium expansum
Phoma diseases, caused for example by Phoma lingam
Phomopsis diseases, caused for example by Phomopsis sojae;
Phytophthora diseases, caused for example by Phytophthora cactorum;
Pyrenophora diseases, caused for example by Pyrenophora graminea
Pyricularia diseases, caused for example by Pyricularia oryzae;
Pythium diseases, caused for example by Pythium ultimum;
Rhizoctonia diseases, caused for example by Rhizoctonia solani;
Rhizopus diseases, caused for example by Rhizopus oryzae
Sclerotium diseases, caused for example by Sclerotium rolfsii;
Septoria diseases, caused for example by Septoria nodorum;
Typhula diseases, caused for example by Typhula incarnate;
Verticillium diseases, caused for example by Verticillium dahliae;

Canker, broom and dieback diseases such as:
Nectria diseases, caused for example by Nectria galligena;

Blight diseases such as:
Monilinia diseases, caused for example by Monilinia laxa;

Leaf blister or leaf curl diseases such as:
Exobasidium diseases caused for example by Exobasidium vexans
Taphrina diseases, caused for example by Taphrina deformans;

Decline diseases of wooden plants such as:
Esca diseases, caused for example by Phaemoniella clamydospora;
Eutypa dyeback, caused for example by Eutypa lata;
Ganoderma diseases caused for example by Ganoderma boninense;
Rigidoporus diseases caused for example by Rigidoporus lignosus

Diseases of Flowers and Seeds such as

Botrytis diseases caused for example by Botrytis cinerea;

Diseases of Tubers such as
Rhizoctonia diseases caused for example by Rhizoctonia solani;

Helminthosporium diseases caused for example by Helminthosporium solani;

Club root diseases such as
Plasmodiophora diseases, cause for example by Plamodiophora brassicae.

Diseases caused by Bacterial Organisms such as
Xanthomonas species for example Xanthomonas campestris pv. oryzae;
Pseudomonas species for example Pseudomonas syringae pv. lachrymans;
Erwinia species for example Erwinia amylovora.

The composition according to the invention may also be used against fungal diseases liable to grow on or inside timber. The term “timber” means all types of species of wood, and all types of working of this wood intended for construction, for example solid wood, high-density wood, laminated wood, and plywood. The method for treating timber according to the invention mainly consists in contacting one or more compounds according to the invention or a composition according to the invention; this includes for example direct application, spraying, dipping, injection or any other suitable means.

The dose of active compound usually applied in the method of treatment according to the invention is generally and advantageously from 10 to 800 g/ha, preferably from 50 to 300 g/ha for applications in foliar treatment. The dose of active substance applied is generally and advantageously from 2 to 200 g per 100 kg of seed, preferably from 3 to 150 g per 100 kg of seed in the case of seed treatment. It is clearly understood that the doses indicated herein are given as illustrative examples of the method according to the invention. A person skilled in the art will know how to adapt the application doses, notably according to the nature of the plant or crop to be treated.

The compounds or mixtures according to the invention can also be used for the preparation of composition useful to curatively or preventively treat human or animal fungal diseases such as, for example, mycoses, dermatoses, trichophyton diseases and candidiases or diseases caused by Aspergillus spp., for example Aspergillus fumigatus.

The preparation and the use of the active compounds I-methyl-3-dihalogenomethyl-5-halogenopyrazole(thio)carboxamides of the formula (I) according to the invention and the intermediates is illustrated by the examples below.

Procedure for Synthesizing Amides of the Formula (I) According to the Invention from Compounds of the Formula (II) and Compounds of the Formula (III):

4 mL of a 0.15 molar solution (0.60 mmol) of an amine according to formula III as described above are initially charged in a 13 mL Chemspeed™ reaction tube in dichloromethane, followed by 0.72 mmol of triethylamine. At a rate of 1 mL/min, 2 mL of a 0.30 molar solution of the acyl chloride (IIb) or (IIe) (0.60 mmol) are added, and the mixture is stirred at room temperature overnight. 1 mL of water is then added, and the mixture is applied to a cartridge with basic alumina (weight 2 g) and eluted with dichloromethane. The solvent is removed and the reaction mixture is analyzed by LCMS and NMR. Impure products are purified further by preparative LCMS.

### Procedure for Synthesizing the Carboxylic Acid Derivatives of the Formula (II) According to the Invention According to Process P1:

### 5-Chloro-3-(difluoromethyl)-1-methyl-1H-pyrazole-4-carboxylic acid (Example IIb)

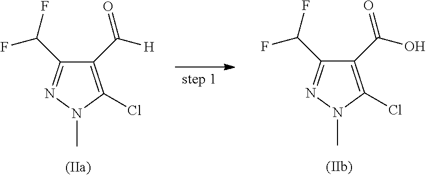

In a 500 mL round-bottom flask, 6.0 g (31 mmol) of 5-chloro-3-(difluoromethyl)-1-methyl-1H-pyrazole-4-carbaldehyde were taken up in 30 mL of toluene. A solution of 2.4 g (62 mmol) of sodium hydroxide in 6 mL of water was added to the reaction mixture, followed by 103 mL of a 30% strength solution of hydrogen peroxide in water. During the addition, the temperature was kept below 37° C. The reaction mixture was then stirred at 50° C. for 7 h. After cooling, the organic phase was extracted with 100 mL of water. The aqueous phase was acidified to pH 2 using dilute hydrochloric acid. The white precipitate formed was filtered off, washed twice with 20 mL of water and dried. This gave 3.2 g of 5-chloro-3-(difluoromethyl)-1-methyl-1H-pyrazole-4-carboxylic acid as a white solid.

¹H NMR (400 MHz, DMSO-d₆) δ ppm: 3.78 (s, 3H); 7.12 (t, 1H, J_{HF}=53.60 Hz); 13.19 (s, 1H);

IR (KBr): 1688 cm⁻¹ (C═O); 2200-3200 cm⁻¹ broad;

### 5-Chloro-3-(difluoromethyl)-1-methyl-1H-pyrazole-4-carbonyl chloride (Example IIc)

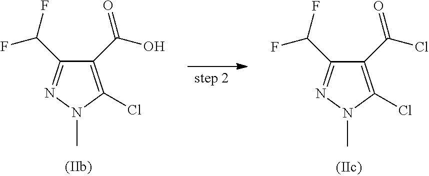

3.2 g of 5-chloro-3-(difluoromethyl)-1-methyl-1H-pyrazole-4-carboxylic acid and 44.3 mL of thionyl chloride were heated under reflux for 5 h. After cooling, the reaction mixture was concentrated under reduced pressure, giving 3.5 g of 5-chloro-3-(difluoromethyl)-1-methyl-1H-pyrazole-4-carbonyl chloride as a yellow oil.

¹H NMR (400 MHz, CHCl₃-d₆) δ ppm: 3.97 (s, 3H); 7.00 (t, J=52.01 Hz, 1H);

IR (TQ): 1759 and 1725 cm⁻¹ (C═O);

### 3-(Difluoromethyl)-5-fluoro-1-methyl-1H-pyrazole-4-carbonyl fluoride (Example IId)

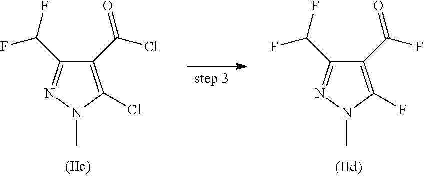

At 100° C., a solution of 5.0 g (22 mmol) of 5-chloro-3-(difluoromethyl)-1-methyl-1H-pyrazole-4-carbonyl chloride in 15 mL of toluene was added to a dried solution of 4.0 g (70 mmol) of potassium fluoride in 21 mL of tetrahydrothiophene-1,1-dioxide. The reaction mixture was then stirred at 190-200° C. for 22 h. Removal of the solvent under reduced pressure gave 8 g of a solution (25% molar) of 3-(difluoromethyl)-5-fluoro-1-methyl-1H-pyrazole-4-carbonyl fluoride in tetrahydrothiophene-1,1-dioxide.

¹H NMR (250 MHz, CHCl₃-d₆) δ ppm: 3.87 (s, 3H); 6.79 (t, J=53.75 Hz, 1H);

¹⁹F NMR (250 MHz, CHCl₃-d₆) δ ppm: 45.37 (s, COF); −117.5 (d, J=28.2 Hz); −131.6 (m);

### 5-Fluoro-3-(difluoromethyl)-1-methyl-1H-pyrazole-4-carboxylic acid (Example IIe)

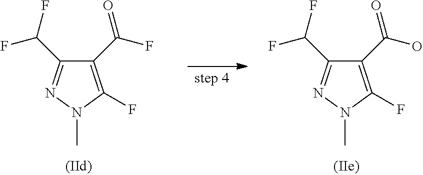

67.5 g of a solution (10% molar) of 3-(difluoromethyl)-5-fluoro-1-methyl-1H-pyrazole-4-carbonyl fluoride in tetrahydrothiophene-1,1-dioxide were added dropwise to 400 mL of an aqueous 1N NaOH solution. During the addition, the temperature was kept below 20° C. After 2 h of stirring at room temperature, the mixture was carefully acidified to pH 2 using concentrated hydrochloric acid. The white precipitate formed was filtered off, washed with water and dried. This gave 6 g of 5-fluoro-3-(difluoromethyl)-1-methyl-1H-pyrazole-4-carboxylic acid as a white solid.

¹H NMR (400 MHz, DMSO-d₆) δ ppm: 3.90 (s, 3H); 7.22 (t, 1H, J_{HF}=53.55 Hz); 13.33 (s, 1H);

### 5-Fluoro-3-(difluoromethyl)-1-methyl-1H-pyrazole-4-carbonyl chloride (Example IIf)

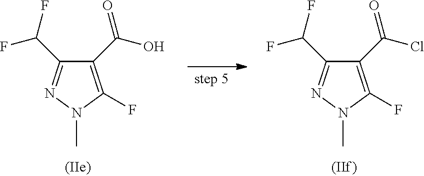

9.1 g of 5-fluoro-3-(difluoromethyl)-1-methyl-1H-pyrazole-4-carboxylic acid and 75.5 mL of thionyl chloride were heated under reflux for 1.5 h. After cooling, the reaction mixture was concentrated under reduced pressure, giving 10 g of 5-fluoro-3-(difluoromethyl)-1-methyl-1H-pyrazole-4-carbonyl chloride as a yellow oil.

GC-MS (M=^{+.})=212; fragments: (M^{+.}-Cl)=177 and (M^{+.}-F)=193;

General Procedure for Synthesizing Thioamides of the Formula (I) According to the Invention from Amides of the Formula (I) According to Process P2:

In a 13 mLChemspeed™ vial is weighed 0.27 mmol of phosphorous pentasulfide (P₂S₅). 3 mL of a 0.18 molar solution of the amide (I) (0.54 mmol) in dioxane is added and the mixture is heated at reflux for two hours. The temperature is then cooled to 80° C. and 2.5 mL of water are added. The mixture is heated at 80° C. for one more hour. 2 mL of water are then added and the reaction mixture is extracted twice by 4 mL of dichloromethane. The organic phase is deposited on a basic alumina cartridge (2 g) and eluted twice by 8 mL of dichloromethane. The solvents are removed and the crude thioamide derivative is analyzed by LCMS and NMR. Insufficiently pure compounds are further purified by preparative LCMS.

The following examples of compounds according to formula (I) are listed in Table 1 below:

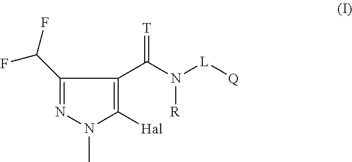

where R represents hydrogen and L has the following meaning:

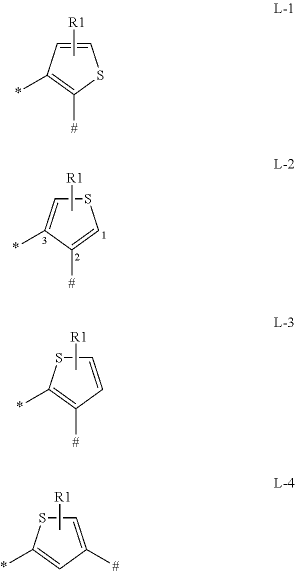

where the bond marked by * is attached to the amide while the bond marked # is attached to Q;

TABLE 1

| | | | | | | | Mass |
|---|---|---|---|---|---|---|---|
| Ex. | Hal | T | L | R¹ | Q | logP | (M + H) |
| 1 | Cl | O | L-1 | H | 3-methylbutyl | 3.74 | 362 |
| 2 | Cl | O | L-1 | H | pentan-2-yl | 3.70 | 362 |
| 3 | Cl | O | L-1 | H | 4-methylpentan-2-yl | 4.19 | 376 |
| 4 | F | O | L-1 | H | 4-methylpentan-2-yl | 3.94 | 360 |
| 5 | Cl | O | L-1 | H | 2-ethylbutyl | 4.03 | 376 |
| 6 | Cl | O | L-1 | H | 3,3-dimethylbutyl | 4.01 | 376 |
| 7 | Cl | O | L-1 | H | 2-hexyl | 4.25 | 376 |
| 8 | Cl | O | L-1 | H | hexan-2-yl | 4.13 | 376 |
| 9 | Cl | O | L-1 | H | 2-methylpentan-3-yl | 4.07 | 376 |
| 10 | Cl | O | L-1 | H | 3-methylpentan-2-yl | 3.99 | 376 |
| 11 | Cl | O | L-1 | H | 5-cyanopentan-2-yl | 2.57 | 387 |
| 12 | Cl | O | L-1 | H | cyclohexylmethyl | 4.27 | 388 |
| 13 | Cl | O | L-1 | H | 2,3-dimethylpentyl | 4.41 | 390 |
| 14 | Cl | O | L-1 | H | heptan-4-yl | 4.54 | 390 |
| 15 | Cl | O | L-1 | H | 5-methylhexan-3-yl | 4.46 | 390 |
| 16 | Cl | O | L-1 | H | 5-methylhexan-2-yl | 4.45 | 390 |
| 17 | Cl | O | L-1 | H | 4,4-dimethylpentan-2-yl | 4.29 | 390 |
| 18 | Cl | O | L-1 | H | 2-(trimethylsilyl)ethyl | 4.30 | |
| 19 | F | O | L-1 | H | 2-(trimethylsilyl)ethyl | 4.06 | |
| 20 | F | S | L-1 | H | 2-(trimethylsilyl)ethyl | 4.46 | 392 |
| 21 | Cl | O | L-1 | H | 1-cyclohexylethyl | 4.56 | 402 |
| 22 | Cl | O | L-1 | H | pent-2-en-2-yl | 3.71 + | 360 |
| | | | | | | 3.87 ⁽¹⁾ | |
| 23 | Cl | O | L-1 | H | 3,3-dimethylbut-1-en- | 3.92 | 374 |
| | | | | | 1-yl | | |
| 24 | Cl | O | L-1 | H | 2-ethylbut-1-en-1-yl | 4.06 | 374 |
| 25 | Cl | O | L-1 | H | hex-1-en-1-yl | 4.12 | 374 |
| 26 | Cl | O | L-1 | H | hex-2-en-2-yl | 4.14 + | 374 |
| | | | | | | 4.27 ⁽¹⁾ | |
| 27 | Cl | O | L-1 | H | 4-methylpent-2-en-3-yl | 4.26 | 374 |
| 28 | Cl | O | L-1 | H | 3-methylpent-2-en-2-yl | 4.23 | 374 |
| 29 | Cl | O | L-1 | H | 5-cyanopent-2-en-2-yl | 2.58 + | 385 |
| | | | | | | 2.70 ⁽¹⁾ | |
| 30 | Cl | O | L-1 | H | cyclohexylidenemethyl | 4.25 | 386 |
| 31 | Cl | O | L-1 | H | 2,3-dimethylpent-1-en- | 4.41 | 388 |
| | | | | | 1-yl | | |
| 32 | Cl | O | L-1 | H | hept-3-en-4-yl | 4.58 + | 388 |
| | | | | | | 4.74 ⁽¹⁾ | |
| 33 | Cl | O | L-1 | H | 4,4-dimethylpent-2-en- | 4.43 + | 388 |
| | | | | | 2-yl | 4.61 ⁽¹⁾ | |
| 34 | Cl | O | L-1 | H | 5-methylhex-2-en-2-yl | 4.46 + | 388 |
| | | | | | | 4.50 ⁽¹⁾ | |
| 35 | Cl | O | L-1 | H | 5-methylhex-3-en-3-yl | 4.51 + | 388 |
| | | | | | | 4.66 ⁽¹⁾ | |
| 36 | Cl | O | L-1 | H | 1-cyclohexylvinyl | 4.78 + | 400 |
| | | | | | | 5.16 | |
| 37 | Cl | O | L-1 | H | 1-phenylprop-1-en-1-yl | 3.70 | 408 |
| 38 | F | O | L-1 | H | phenyl | 3.11 | 352 |
| 39 | F | O | L-2 | 1-trifluoro | phenyl | 4.11 | 420 |
| | | | | methyl | | | |
| 40 | F | O | L-3 | H | 4-methylphenyl | 3.60 | 366 |
| 41 | F | O | L-4 | H | 4-chlorophenyl | 3.87 | 386 |

Note
¹ mixture of isomers

In table 1, unless otherwise specified, M+H (ApcI+) means the molecular ion peak plus 1 a.m.u. (atomic mass unit) as observed in mass spectroscopy via positive atmospheric pressure chemical ionisation.

In table 1, the logP values were determined in accordance with EEC Directive 79/831 Annex V.A8 by HPLC (High Performance Liquid Chromatography) on a reversed-phase column (C 18), using the method described below:

Temperature: 40° C.; Mobile phases: 0.1% aqueous formic acid and acetonitrile; linear gradient from 10% acetonitrile to 90% acetonitrile.

Calibration was carried out using unbranched alkan-2-ones (comprising 3 to 16 carbon atoms) with known logP values (determination of the logP values by the retention times using linear interpolation between two successive alkanones). lambda-max-values were determined using UV-spectra from 200 nm to 400 nm and the peak values of the chromatographic signals.

### NMR Data of Selected Examples

### NMR Peak List Method

The ¹H-NMR data of selected examples are stated in the form of ¹H-NMR peak lists. For each signal peak, the δ value in ppm and the signal intensity in brackets are listed:

### Example 3

¹H NMR (600 MHz, CD3CN-d) δ ppm: 0.84-0.86 (m, 3H); 1.22-1.23 (m, 2H); 1.44-1.55 (m, 2H); 1.93-1.97 (m, 6H); 2.15 (s, 1H); 3.20-3.24 (m, 1H); 3.90 (s, 2H); 7.10 (t, 1H); 7.28 (s, 1H); 8.03 (b, 1H)

### Example 4

¹H NMR (600 MHz, CD3CN-d) δ ppm: 0.84-0.87 (m, 6H); 1.18-1.27 (m, 2H); 1.41-1.52 (m, 2H); 1.92-1.97 (m, 6H); 2.15 (s, 1H); 3.16-3.34 (m, 1H); 3.79 (s, 2H); 7.10 (t, 1H); 7.14 (s, 1H); 7.82 (b, 1H)

### USE EXAMPLES

### Example A Alternaria Test (Tomato)/Preventive

Solvent: 49 parts by weight of N,N-dimethylformamide
Emulsifier: 1 part by weight of alkylarylpolyglycolether

To produce a suitable preparation of active compound, 1 part by weight of active compound is mixed with the stated amounts of solvent and emulsifier, and the concentrate is diluted with water to the desired concentration.

To test for preventive activity, young plants are sprayed with the preparation of active compound at the stated rate of application. One day after this treatment, the plants are inoculated with an aqueous spore suspension of Alternaria solani. The plants remain for one day in an incubation cabinet at approximately 22° C. and a relative atmospheric humidity of 100%. Then the plants are placed in an incubation cabinet at approximately 20° C. and a relative atmospheric humidity of 96%.

The test is evaluated 7 days after the inoculation. 0% means an efficacy which corresponds to that of the untreated control while an efficacy of 100% means that no disease is observed.

In this test, the following compounds from table A according to the invention, show at an active compound concentration of 500 ppm, an efficacy of 70% or more:

TABLE A

| Example | Efficacy |
|---|---|
| 1 | 100 |
| 2 | 94 |
| 3 | 100 |
| 4 | 100 |
| 5 | 100 |
| 6 | 95 |
| 8 | 94 |
| 9 | 94 |
| 12 | 100 |
| 13 | 95 |
| 15 | 88 |
| 16 | 88 |
| 17 | 94 |
| 18 | 80 |
| 19 | 90 |
| 21 | 95 |
| 22 | 94 |
| 23 | 70 |
| 26 | 75 |
| 27 | 75 |
| 28 | 94 |
| 30 | 70 |
| 33 | 88 |

### Example B

### Sphaerotheca Test (Cucumber)/Preventive

Solvent: 49 parts by weight of N,N-dimethylformamide
Emulsifier: 1 part by weight of alkylarylpolyglycolether

To produce a suitable preparation of active compound, 1 part by weight of active compound is mixed with the stated amounts of solvent and emulsifier, and the concentrate is diluted with water to the desired concentration.

To test for preventive activity, young plants are sprayed with the preparation of active compound at the stated rate of application. One day after this treatment, the plants are inoculated with an aqueous spore suspension of Sphaerotheca fuliginea. Then the plants are placed in a greenhouse at approximately 23° C. and a relative atmospheric humidity of approximately 70%.

The test is evaluated 7 days after the inoculation. 0% means an efficacy which corresponds to that of the untreated control, while an efficacy of 100% means that no disease is observed.

In this test, the following compounds from table B according to the invention, show at an active compound concentration of 500 ppm, an efficacy of 85% or more:

TABLE B

| Example | Efficacy |
|---|---|
| 3 | 96 |
| 4 | 100 |
| 6 | 89 |
| 17 | 100 |
| 18 | 98 |
| 19 | 100 |

### Example C

### Venturia Test (Apples)/Preventive

Solvent: 24.5 parts by weight of acetone
   24.5 parts by weight of N,N-dimethylformamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for preventive activity, young plants are sprayed with the preparation of active compound at the stated rate of application. After the spray coating has dried on, the plants are inoculated with an aqueous conidia suspension of the causal agent of apple scab (Venturia inaequalis) and then remain for 1 day in an incubation cabinet at approximately 20° C. and a relative atmospheric humidity of 100%.

The plants are then placed in a greenhouse at approximately 21° C. and a relative atmospheric humidity of approximately 90%.

The test is evaluated 10 days after the inoculation. 0% means an efficacy which corresponds to that of the untreated control, while an efficacy of 100% means that no disease is observed.

In this test, the following compounds from table C according to the invention, show at an active compound concentration of 100 ppm, an efficacy of 90% or more:

TABLE C

| Example | Efficacy |
|---|---|
| 3 | 100 |
| 4 | 100 |
| 18 | 94 |
| 19 | 99 |

### Example D

### Uromyces Test (Beans)/Preventive

Solvent: 24.5 parts by weight of acetone
   24.5 parts by weight of N,N-dimethylformamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for preventive activity, young plants are sprayed with the preparation of active compound at the stated rate of application. After the spray coating has dried on, the plants are inoculated with an aqueous spore suspension of the causal agent of bean rust (Uromyces appendiculatus) and then remain for 1 day in an incubation cabinet at approximately 20° C. and a relative atmospheric humidity of 100%.

The plants are then placed in a greenhouse at approximately 21° C. and a relative atmospheric humidity of approximately 90%.

The test is evaluated 10 days after the inoculation. 0% means an efficacy which corresponds to that of the untreated control, while an efficacy of 100% means that no disease is observed.

In this test, the following compounds from table D according to the invention, show at an active compound concentration of 100 ppm, a total efficacy:

TABLE D

| Example | Efficacy |
|---|---|
| 3 | 100 |
| 4 | 100 |
| 18 | 100 |
| 19 | 100 |

### Example E

### Botrytis Test (Beans)/Preventive

Solvent: 24.5 parts by weight of acetone
   24.5 parts by weight of N,N-dimethylformamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for preventive activity, young plants are sprayed with the preparation of active compound. After the spray coating has dried on, 2 small pieces of agar covered with growth of Botrytis cinerea are placed on each leaf. The inoculated plants are placed in a darkened chamber at 20° C. and a relative atmospheric humidity of 100%.

2 days after the inoculation, the size of the lesions on the leaves is evaluated. 0% means an efficacy which corresponds to that of the untreated control, while an efficacy of 100% means that no disease is observed.

In this test, the following compounds from table E according to the invention, show at an active compound concentration of 100 ppm, an efficacy of 90% or more:

TABLE E

| Example | Efficacy |
|---|---|
| 3 | 98 |
| 4 | 97 |
| 18 | 91 |
| 19 | 100 |

### Example F

### Blumeria Test (Barley)/Preventive

Solvent: 49 parts by weight of N,N-dimethylacetamid
Emulsifier: 1 part by weight of alkylaryl polyglycol ether

To produce a suitable preparation of active compound, 1 part by weight of active compound or active compound combination is mixed with the stated amounts of solvent and emulsifier, and the concentrate is diluted with water to the desired concentration.

To test for preventive activity, young plants are sprayed with the preparation of active compound or active compound combination at the stated rate of application. After the spray coating has been dried, the plants are dusted with spores of Blumeria graminis f.sp. hordei.

The plants are placed in the greenhouse at a temperature of approximately 18° C. and a relative atmospheric humidity of approximately 80% to promote the development of mildew pustules.

The test is evaluated 7 days after the inoculation. 0% means an efficacy which corresponds to that of the untreated control, while an efficacy of 100% means that no disease is observed.

In this test, the following compounds from table F according to the invention, show at an active compound concentration of 500 ppm, an efficacy of 90% or more:

TABLE F

| Example | Efficacy |
|---|---|
| 1 | 94 |
| 3 | 100 |
| 4 | 100 |
| 6 | 94 |
| 12 | 94 |
| 13 | 100 |
| 15 | 94 |
| 18 | 92 |
| 19 | 100 |

### Example G

### Fusarium nivale (Var. Majus)-Test (Wheat)/Preventive

Solvent: 49 parts by weight of N,N-dimethylacetamide
Emulsifier: 1 part by weight of alkylaryl polyglycol ether

To produce a suitable preparation of active compound, 1 part by weight of active compound or active compound combination is mixed with the stated amounts of solvent and emulsifier, and the concentrate is diluted with water to the desired concentration.

To test for preventive activity, young plants are sprayed with the preparation of active compound or active compound combination at the stated rate of application.

After the spray coating has been dried, the plants are slightly injured by using a sandblast and afterwards they are sprayed with a conidia suspension of Fusarium nivale (var. majus).

The plants are placed in the greenhouse under a translucent incubation cabinet at a temperature of approximately 10° C. and a relative atmospheric humidity of approximately 100%.

The test is evaluated 5 days after the inoculation. 0% means an efficacy which corresponds to that of the untreated control, while an efficacy of 100% means that no disease is observed.

In this test, the following compounds from table G according to the invention, show at an active compound concentration of 500 ppm, an efficacy of 80% or more:

TABLE G

| Example | Efficacy |
|---|---|
| 1 | 80 |
| 3 | 100 |
| 6 | 100 |
| 7 | 90 |
| 13 | 100 |
| 18 | 100 |
| 19 | 100 |

Under the same conditions, high (at least 80%) protection to total protection is observed at a dose of 250 ppm and 500 ppm of active ingredient with compound 3, whereas poor (less than 15%) protection to average (less than 50%) protection is observed with the compound of example 1.13 disclosed in patent application EP-0737682 as in table G2.

TABLE G2

| Example | dose (ppm) | Efficacy |
|---|---|---|
| 3 from this invention | 500 | 100 |
| | 250 | 86 |
| 1.13 from EP-0737682 | 500 | 43 |
| | 250 | 14 |

Example 1.13 disclosed in international patent EP-0737682 corresponds to 1-methyl-N-[2-(4-methylpentan-2-yl)-3-thienyl]-3-(trifluoromethyl)-1H-pyrazole-4-carboxamide [commun name: penthiopyrad].

These results show that the compounds according to the invention have a better biological activity than the structurally closest compounds disclosed in EP-0737682.

### Example H

### Puccinia Test (Wheat)/Preventive

Solvent: 49 parts by weight of N,N-dimethylformamide
Emulsifier: 1 part by weight of alkylarylpolyglycolether

To produce a suitable preparation of active compound, 1 part by weight of active compound is mixed with the stated amounts of solvent and emulsifier, and the concentrate is diluted with water to the desired concentration.

To test for preventive activity, young plants are sprayed with the preparation of active compound at the stated rate of application. One day after this treatment, the plants are inoculated with an aqueous spore suspension of Puccinia recondita. The plants remain for 48 hours in an incubation cabinet at 22° C. and a relative atmospheric humidity of 100%. Then the plants are placed in a greenhouse at a temperature of approximately 20° C. and a relative atmospheric humidity of approximately 80%.

The test is evaluated 7-9 days after the inoculation. 0% means an efficacy which corresponds to that of the untreated control while an efficacy of 100% means that no disease is observed.

In this test, the following compounds from table H according to the invention, show at an active compound concentration of 500 ppm, an efficacy of 70% or more:

TABLE H

| Example | Efficacy |
|---|---|
| 1 | 95 |
| 2 | 100 |
| 3 | 100 |
| 4 | 100 |
| 5 | 90 |
| 6 | 100 |
| 7 | 100 |
| 8 | 95 |
| 9 | 80 |
| 10 | 90 |
| 11 | 100 |
| 12 | 95 |
| 13 | 90 |
| 14 | 70 |
| 15 | 90 |
| 16 | 100 |
| 17 | 100 |
| 18 | 100 |
| 19 | 100 |
| 21 | 95 |
| 22 | 80 |
| 23 | 90 |
| 24 | 90 |
| 27 | 80 |
| 28 | 80 |
| 32 | 80 |
| 33 | 95 |
| 36 | 80 |

### Example I

### Pyrenophora Test (Barley)/Preventive

Solvent: 49 parts by weight of N,N-dimethylformamide
Emulsifier: 1 part by weight of alkylarylpolyglycolether

To produce a suitable preparation of active compound, 1 part by weight of active compound is mixed with the stated amounts of solvent and emulsifier, and the concentrate is diluted with water to the desired concentration.

To test for preventive activity, young plants are sprayed with the preparation of active compound at the stated rate of application. One day after this treatment, the plants are inoculated with an aqueous spore suspension of Pyrenophora teres. The plants remain for 48 hours in an incubation cabinet at 22° C. and a relative atmospheric humidity of 100%. Then the plants are placed in a greenhouse at a temperature of approximately 20° C. and a relative atmospheric humidity of approximately 80%.

The test is evaluated 7-9 days after the inoculation. 0% means an efficacy which corresponds to that of the untreated control while an efficacy of 100% means that no disease is observed.

In this test, the following compounds from table I according to the invention, show at an active compound concentration of 500 ppm, an efficacy of 70% or more:

TABLE I

| Example | Efficacy |
|---|---|
| 1 | 100 |
| 2 | 100 |
| 3 | 100 |
| 4 | 100 |
| 5 | 100 |
| 6 | 100 |
| 7 | 95 |
| 8 | 100 |
| 9 | 100 |
| 10 | 100 |
| 11 | 94 |
| 12 | 100 |
| 13 | 100 |
| 14 | 94 |
| 15 | 100 |
| 16 | 94 |
| 17 | 94 |
| 18 | 100 |
| 19 | 100 |
| 21 | 100 |
| 22 | 94 |
| 23 | 80 |
| 24 | 90 |
| 26 | 89 |
| 27 | 89 |
| 28 | 100 |
| 30 | 80 |
| 31 | 70 |
| 33 | 94 |
| 34 | 78 |
| 36 | 95 |

### Example J

### Pyricularia Test (Rice)/Protective

Solvent: 28.5 parts by weight of acetone
Emulsifier: 1.5 part by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. 1 day after the treatment, the plants are inoculated with an aqueous spore suspension of Pyricularia oryzae. The plants are then placed in a greenhouse at a relative atmospheric humidity of 100% and a temperature of 25° C.

Evaluation is carried out 5 days after the inoculation. 0% means an efficacy which corresponds to that of the control, whereas an efficacy of 100% means that no infection is observed.

In this test, the following compound according to the invention, show at an active compound concentration of 250 ppm, an efficacy of 95% or more:

Example no. 3 (97%).

### Example K

### Rhizoctonia Test (Rice)/Protective

Solvent: 28.5 parts by weight of acetone
Emulsifier: 1.5 part by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. 1 day after the treatment, the plants are inoculated with hyphae of Rhizoctonia solani. The plants are then placed in a greenhouse at a relative atmospheric humidity of 100% and a temperature of 25° C.

Evaluation is carried out 4 days after the inoculation. 0% means an efficacy which corresponds to that of the control, whereas an efficacy of 100% means that no infection is observed.

In this test, the following compound according to the invention, show at an active compound concentration of 250 ppm, an efficacy of 95% or more:

Example no. 3 (100%).

### Example L

### Cochliobolus Test (Rice)/Protective

Solvent: 28.5 parts by weight of acetone
Emulsifier: 1.5 part by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. 1 day after the treatment, the plants are inoculated with an aqueous spore suspension of Cochliobolus miyabeanus. The plants are then placed in a greenhouse at a relative atmospheric humidity of 100% and a temperature of 25° C.

Evaluation is carried out 4 days after the inoculation. 0% means an efficacy which corresponds to that of the control, whereas an efficacy of 100% means that no infection is observed.

In this test, the following compound according to the invention, show at an active compound concentration of 250 ppm, an efficacy of 95% or more:

Example no. 3 (97%).

### Example M

### Phakopsora Test (Soybeans)/Protective

Solvent: 28.5 parts by weight of acetone
Emulsifier: 1.5 parts by weight of polyoxyethylene alkyl phenyl ether

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated rate of application. One day after spraying, the plants are inoculated with an aqueous spore suspension of the causal agent of soybean rust (Phakopsora pachyrhizi). The plants are then placed in a greenhouse at approximately 20° C. and a relative atmospheric humidity of approximately 80%.

The test is evaluated 11 days after the inoculation. 0% means an efficacy which corresponds to that of the control, while an efficacy of 100% means that no disease is observed.

In this test, the following compounds from table M according to the invention, show at an active compound concentration of 250 ppm, an efficacy of 95% or more:

TABLE M

| Example | Efficacy |
|---|---|
| 3 | 98 |
| 4 | 99 |

1. A 1-methyl-3-dihalogenomethyl-5-halogenopyrazole(thio)carboxamide compound of formula (I) 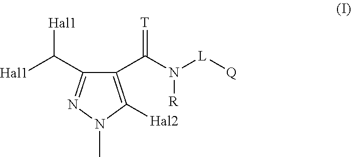
in which
T represents an oxygen or sulfur atom
R represents hydrogen, C₁-C₆-alkylsulfonyl, C₁-C₄-alkoxy-C₁-C₄-alkyl, C₁-C₄-haloalkylsulfonyl, halo-C₁-C₄-alkoxy-C₁-C₄-alkyl or formyl;
Hal1 and Hal2 independently of one another represent chlorine or fluorine;
Q represents Q¹, Q², Q³, Q⁴, Q⁵, or Q⁶;
L represents 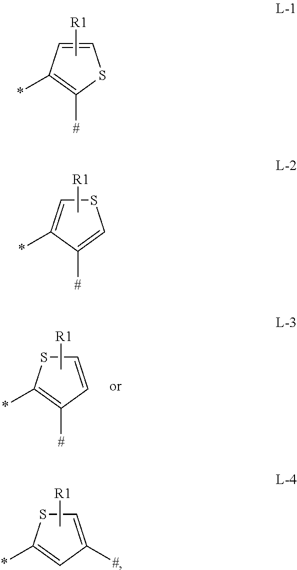
where the bond marked by * is attached to the amide while the bond marked # is attached to Q;
R¹ represents hydrogen, halogen, C₁-C₄-alkyl or C₁-C₄-haloalkyl;
Q¹ represents 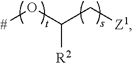
where the bond marked # is attached to L;
R² represents hydrogen, C₁-C₄-alkyl, C₃-C₆-cycloalkyl, C₁-C₄-haloalkyl or optionally substituted phenyl;
Z¹ represents —CR³R⁴R⁵ or —SiR³R⁴R⁵;
s represents 0, 1, 2 or 3;
t represents 0 or 1;
R³ represent hydrogen, cyano, C₁-C₈-alkyl, or C₁-C₆-haloalkyl;
R⁴, R⁵ independently of one another represents hydrogen, C₁-C₈-alkyl, C₁-C₆-haloalkyl; or
R³ and R⁴ together with the carbon atom to which said R³ and R⁴ are attached form a 3- to 6-membered optionally substituted carbocyclic or heterocyclic saturated or unsaturated ring;
Q² represents 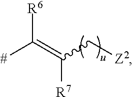
where the bond marked # is attached to L;
R⁶ represents hydrogen, C₁-C₄-alkyl, C₃-C₆-cycloalkyl, C₁-C₄-haloalkyl or optionally substituted phenyl;
R⁷ represents hydrogen, C₁-C₄-alkyl, or C₁-C₄-haloalkyl;
Z² represents hydrogen, —CR⁸R⁹R¹⁰ or —SiR⁸R⁹R¹⁰;
u represents 0, 1 or 2;
R⁸ represent hydrogen, cyano, C₁-C₈-alkyl, or C₁-C₆-haloalkyl; or
R⁷ and R⁸ together with the carbon atoms to which said R⁷ and R⁸ are attached form a 3- to 6-membered optionally substituted carbocyclic saturated or unsaturated ring;
R⁹, R¹⁰ independently of one another represent hydrogen, C₁-C₈-alkyl, or C₁-C₆-haloalkyl; or
R⁸ and R⁹ together with the carbon atom to which said R⁸ and R⁹ are attached form a 3- to 6-membered optionally substituted carbocyclic saturated or unsaturated ring;
Q³ represents 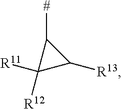
where the bond marked # is attached to L;
R¹¹ represents hydrogen or halogen;
R¹² represents hydrogen or halogen;
R¹³ represents optionally substituted C₂-C₁₂-alkyl, optionally substituted C₂-C₁₂-alkenyl, optionally substituted C₂-C₁₂-alkynyl, optionally substituted C₃-C₁₂-cycloalkyl, optionally substituted phenyl or heterocyclyl;
Q⁴ represents 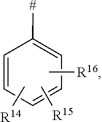
where the bond marked # is attached to L,
R¹⁴, R¹⁵ and R¹⁶ independently of one another represent halogen, cyano, nitro, C₁-C₆-alkyl, C₂-C₆-alkenyl, C₁-C₄-alkoxy, C₁-C₄-alkylsulfanyl, C₁-C₄-alkylsulfonyl, C₃-C₆-cycloalkyl, or represent C₁-C₄-haloalkyl, C₁-C₄-haloalkoxy, C₁-C₄-haloalkylsulfanyl or C₁-C₄-haloalkylsulfonyl having in each case 1 to 5 halogen atoms;
Q⁵ represents 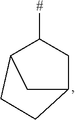
where the bond marked # is attached to L;
Q⁶ represents 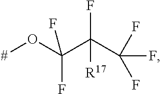
where R¹⁷ represents hydrogen or fluorine;
where, unless indicated otherwise, a group or a substituent which is substituted according to the invention is substituted by one or more group selected in the list consisting of halogen; nitro, cyano, C₁-C₁₂-alkyl; C₁-C₆-haloalkyl having from 1 to 9 identical or different halogen atoms; C₁-C₆-alkoxy; C₁-C₆-haloalkoxy having from 1 to 9 identical or different halogen atoms; C₁-C₆-alkylsulfanyl; C₁-C₆-haloalkylsulfanyl having from 1 to 9 identical or different halogen atoms; C₁-C₆-alkylsulfonyl; C₁-C₆-haloalkylsulfonyl having from 1 to 9 identical or different halogen atoms; C₂-C₁₂-alkenyl; C₂-C₁₂-alkynyl; C₃-C₇-cycloalkyl; phenyl; tri(C₁-C₈)alkylsilyl; tri(C₁-C₈)alkylsilyl-C₁-C₈-alkyl.

2. A compound according to claim 1, wherein T represents an oxygen atom.

3. A compound according to claim 1, wherein R represents hydrogen, methylsulfonyl, ethylsulfonyl, n- or isopropylsulfonyl, n-, iso-, sec- or tert-butylsulfonyl, methoxymethyl, methoxyethyl, ethoxymethyl, ethoxyethyl, trifluoromethylsulfonyl, trifluoromethoxymethyl or formyl.

4. A compound according to claim 1, wherein R represents hydrogen, methoxymethyl, or formyl.

5. A compound according to claim 1, wherein R¹ represents hydrogen or chlorine.

6. A compound according to claim 1, wherein s or u represents 1.

7. A compound according to claim 1, wherein R² and R¹⁰ independently of one another represents hydrogen, fluorine, chlorine, methyl, ethyl, trifluoromethyl, difluoromethyl, fluoromethyl, trichloromethyl, dichloromethyl, chloromethyl, chlorofluoromethyl, fluorodichloromethyl, difluorochloromethyl, pentafluoroethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2-chloro-2,2-difluoroethyl, 2-dichloro-2-fluoroethyl, 2,2,2-trichloroethyl, 1-chlorobutyl, heptafluoro-n-propyl or heptafluoroisopropyl.

8. A compound according to claim 1, wherein R⁵ represents represents hydrogen, fluorine, chlorine, methyl, ethyl, n- or isopropyl, iso-, sec- or tert-butyl, trifluoromethyl, difluoromethyl, fluoromethyl, trichloromethyl, dichloromethyl, chloromethyl, chlorofluoromethyl, fluorodichloromethyl, difluorochloromethyl, pentafluoroethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2-chloro-2,2-difluoroethyl, 2-dichloro-2-fluoroethyl, 2,2,2-trichloroethyl, 1-chlorobutyl, heptafluoro-n-propyl or heptafluoroisopropyl.

9. A compound according to claim 1, wherein R³, R⁴, R⁸ and R⁹ independently of one another represent fluorine, chlorine, methyl, ethyl, n- or isopropyl, n-, iso-, sec- or tert-butyl, trifluoromethyl, difluoromethyl, fluoromethyl, trichloromethyl, dichloromethyl, chloromethyl, chlorofluoromethyl, fluorodichloromethyl, difluorochloromethyl, pentafluoroethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2-chloro-2,2-difluoroethyl, 2-dichloro-2-fluoroethyl, 2,2,2-trichloroethyl, 1-chlorobutyl, heptafluoro-n-propyl or heptafluoroisopropyl.

10. A compound according to claim 1, wherein R³ and R⁴ together with the carbon atom to which said R³ and R⁴ are attached, or R⁸ and R⁹ together with the carbon atom to which said R⁸ and R⁹ are attached, form a 3- to 6-membered carbocyclic or heterocyclic saturated or unsaturated ring which is optionally substituted by halogen, methyl, ethyl, methoxy, trifluoromethyl or trifluoromethoxy,

11. A compound according to claim 1, wherein R⁶ and R⁷ independently of one another represents hydrogen, methyl or ethyl.

12. A compound according to claim 1, wherein R¹³ represents C₂-C₆-alkyl, optionally substituted C₃-C₈-cycloalkyl, optionally substituted phenyl, pyridyl, thienyl or furyl.

13. A compound according to claim 1, wherein R¹⁴, R¹⁵ and R¹⁶ independently of one another represent fluorine, chlorine, bromine, cyano, nitro, methyl, ethyl, n- or isopropyl, n-, i-, s- or t-butyl, methoxy, ethoxy, methylsulfanyl, ethylsulfanyl, n- or isopropylsulfanyl, cyclopropyl, trifluoromethyl, trichloromethyl, trifluoroethyl, difluoromethoxy, trifluoromethoxy, difluorochloromethoxy, trifluoroethoxy, difluoromethylsulfanyl, difluorochloromethylsulfanyl or trifluoromethylsulfanyl.

14. A compound according to claim 1, wherein:
T represents oxygen;
R represents hydrogen;
Hal represents chlorine or fluorine;
Hal1 and Hal2 independently of one another represent chlorine or fluorine;
Q represents Q¹, Q², Q³, Q⁴, Q⁵, or Q⁶;
L represents, 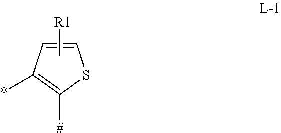
where the bond marked * is attached to the amide while the bond marked # is attached to Q;
R¹ represents hydrogen;
Q¹ represents 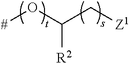
where the bond marked # is attached to L,
R² represents hydrogen, methyl or ethyl,
Z¹ represents —CR³R⁴R⁵ or —SiR³R⁴R⁵;
s represents 0, 1, 2 or 3;
t represents 0,
R³, R⁴, R⁵ independently of one another represent hydrogen or methyl;
Q² represents 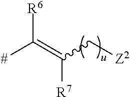
where the bond marked # is attached to L,
R⁶ represents hydrogen or methyl,
R⁷ represents hydrogen or methyl,
Z² represents —CR⁸R⁹R¹⁰ or —SiR⁸R⁹R¹⁰;
u represents 0, 1 or 2;
R⁸, R⁹, R¹⁰ independently of one another represent hydrogen or methyl;
Q³ represents 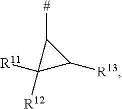
where the bond marked # is attached to L;
R¹¹ and R¹² represent hydrogen;
R¹³ represents cyclopropyl;
Q⁴ represents 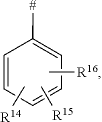
where the bond marked # is attached to L;
R¹⁴, R¹⁵ and R¹⁶ independently of one another represent fluorine, chlorine, bromine or trifluoromethyl;
Q⁵ represents 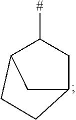
Q⁶ represents

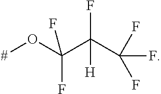

15. A fungicide composition comprising, as an active ingredient, an effective amount of a compound of formula (I) according to claim 1, and an agriculturally acceptable support, carrier and/or filler.

16. A method for controlling phytopathogenic fungi of crops,
comprising applying an agronomically effective and substantially non-phytotoxic quantity of a compound according to claim 1,
to soil where a plant grows or is capable of growing, to leaves and/or fruit of a plant and/or to seeds of a plant.

17. A compound according to claim 2, wherein R represents hydrogen, methylsulfonyl, ethylsulfonyl, n- or isopropylsulfonyl, n-, iso-, sec- or tert-butylsulfonyl, methoxymethyl, methoxyethyl, ethoxymethyl, ethoxyethyl, trifluoromethylsulfonyl, trifluoromethoxymethyl or formyl.

18. A method for controlling phytopathogenic fungi of crops, comprising applying a fungicide composition according to claim 15, is applied to soil where a plant grows or is capable of growing, to leaves and/or fruit of a plans and/or to seeds of a plant.
